(19) United States
(12) Patent Application Publication
(10) Pub. No.: US 2024/0024032 A1
(43) Pub. Date: Jan. 25, 2024

(54) TRACKING AN IMPLANTABLE DEVICE POWERED USING ULTRASONIC WAVES

(71) Applicant: Iota Biosciences, Inc., Alameda, CA (US)

(72) Inventors: Joshua KAY, Oakland, CA (US); Suraj GOWDA, Moraga, CA (US); Michel M. MAHARBIZ, El Cerrito, CA (US); Jose M. CARMENA, Alameda, CA (US)

(73) Assignee: Iota Biosciences, Inc., Alameda, CA (US)

(21) Appl. No.: 18/022,969

(22) PCT Filed: Aug. 24, 2021
(86) PCT No.: PCT/US2021/047353

§ 371 Date: Feb. 23, 2023

Related U.S. Application Data
(60) Provisional application No. 63/069,522, filed on Aug. 24, 2020.

(51) Int. Cl. A61B 34/20; A61B 8/08; A61N 1/378; A61N 1/372; H02J 50/15; H02J 50/90

(52) U.S. Cl. CPC A61B 34/20; A61B 8/0833; A61N 1/3787; A61N 1/37217; H02J 50/15; H02J 50/90; A61B 2034/2063

(57) ABSTRACT

Method and system embodiments for discovering or tracking a device implantable in a subject using ultrasonic waves are described. The method for tracking the implantable device can include establishing a synchronization state with the implantable device, estimating a location of the implantable device, and determining whether to maintain or to adjust were an ultrasonic beam is being focused based on ultrasonic signal strength. The method for discovering an implantable device powered using ultrasonic waves can include emitting an ultrasonic beam to successively focus on a plurality of focal points, receiving ultrasonic backscatter corresponding to the ultrasonic beam focused on the focal point, and comparing the received ultrasonic backscatter with a predetermined pattern associated with the implantable device to be discovered to generate a score indicating how likely the ultrasonic backscatter comprises the predetermined pattern; and determining a location of the implantable device from based on the scores.

### CROSS-REFERENCE TO RELATED APPLICATION

This application claims the priority benefit to U.S. Provisional Application No. 63/069,522, filed Aug. 24, 2020, which is incorporated herein by reference for purposes.

### FIELD OF THE DISCLOSURE

The present disclosure relates generally to powering an implantable device using ultrasonic waves and, more specifically, to tracking the implantable device using ultrasonic waves to effectively deliver power to the implantable device.

### BACKGROUND OF THE DISCLOSURE

Methods have been developed for treating various medical conditions of a patient. These methods may involve inserting an implantable medical device such as a cardiac or neural bio-implant within the patient's body. Operating such implantable devices wirelessly fashion continues to be a technical challenge for many biomedical applications. This is, in part, because the traditional approach of using radio frequencies (RF) to control wireless devices has many limitations in the biomedical context and may pose a health hazard to the patient. For example, an RF antenna needed to process RF may have a large form factor and would render the implantable device using the RF antenna too large to be safely and comfortably placed at many locations in the body. Biological tissue also tends to easily absorb energy from RF carrier frequencies, which may limit the implantable depth of the implantable device. In addition, due to the high absorption rate of RF energy, biological tissue may more likely overheat and pose a health hazard to the patient.

One alternative to using RF is to use external ultrasound interrogators that emit ultrasonic waves to operate and power small implantable devices within the patient. During use, however, the interrogator and a targeted implantable device are often not in alignment due to motion between the interrogator and the implantable device. For example, due to body motion or the patient's breathing, the position of the implantable device may shift. Similarly, due to the interrogator operator's motion (e.g., shaky hands or body motion), the position of the interrogator may shift. In either case, the interrogator may not be efficiently powering the implantable device due to the misalignment. Although the power delivered by the interrogator can be increased to compensate for the misalignment, the ultrasound power can only be increased so much to stay within regulatory guidelines and to prevent harming the patient's body. If the implantable device is not tracked efficiently, the implantable device may not be sufficiently powered and its operations may be unreliable.

The disclosures of all publications, patents, and patent applications referred to herein are each hereby incorporated by reference in their entireties. To the extent that any reference incorporated by reference conflicts with the instant disclosure, the instant disclosure shall control.

### SUMMARY OF THE DISCLOSURE

The use of ultrasonic waves to operate and power an implantable device can be advantageous over other approaches because biological tissues have significantly lower absorption rates of ultrasonic waves than other types of waves such as RF waves. This property of ultrasonic waves can allow the device to be implantable at greater depths in the subject as well as to reduce tissue heating due to energy absorbed by the tissue. For example, an implantable device can include an ultrasonic transducer capable of receiving ultrasonic waves emitted by the interrogator and converting the mechanical energy of the received ultrasonic waves into electrical energy to power the implantable device. There remains a need, however, to enable an interrogator to efficiently track implantable devices powered using ultrasonic waves.

In some embodiments, a method for tracking an implantable device powered using ultrasonic waves to maintain power supplied to the implantable device comprises establishing a synchronization state with the implantable device, comprising: emitting an ultrasonic beam to a first focal point and receiving a first ultrasonic backscatter corresponding to the emitted ultrasonic beam; determining a first signal strength based on the first ultrasonic backscatter; and establishing the synchronization state with the implantable device in response to determining that the first signal strength is at or above a predetermined threshold; estimating a location of the implantable device; emitting the ultrasonic beam to a second focal point closer to the estimated location than the first focal point and receiving second ultrasonic backscatter corresponding to the emitted ultrasonic beam; determining a second signal strength based on the second ultrasonic backscatter; and determining whether to maintain or to adjust where the emitted ultrasonic beam is being focused based on comparing the determined second signal strength with the first signal strength.

In some embodiments of the method for tracking an implantable device, establishing the synchronization state comprises controlling the ultrasonic beam to successively focus on a plurality of focal points in a search region to determine the first focal point at which the first signal strength meets the predetermined threshold. In some embodiments, controlling the ultrasonic beam comprises directing the ultrasonic beam in a first direction to successively focus on the plurality of focal points until the first signal strength determined from the first ultrasonic backscatter is determined to be above the predetermined threshold.

In some embodiments of the method for tracking an implantable device, the method comprises, in response to determining to maintain the determined focal point of the ultrasonic beam at the second focal point maintaining the ultrasonic beam to focus on the determined second focal point, and monitoring a signal strength determined from ultrasonic backscatter received while the ultrasonic beam is focused on the determined second focal point.

In some embodiments, the monitored signal strength corresponds to a modulated signal generated by the implantable device to encode information into ultrasonic backscatter received at an interrogator. In some embodiments, the encoded information uniquely identifies the implantable device.

In some embodiments of the method for tracking an implantable device, the method comprises, in response to determining to adjust the second focal point of the ultrasonic beam, iteratively estimating the location of the implantable device based on received ultrasonic backscatter and updating a focal point of the ultrasonic beam in the direction of the estimated location until a signal strength determined from ultrasonic backscatter received for the updated focal point is no longer increasing.

In some embodiments of the method for tracking an implantable device, determining the first signal strength based on the first ultrasonic backscatter comprises extracting, from the first ultrasonic backscatter, an implant signal associated with the implantable device; and determining the first signal strength based on the extracted implant signal. In some embodiments, extracting the implant signal comprises cancelling signal interferences from the backscattered ultrasonic waves to extract the implant signal. In some embodiments, the method comprises identifying the implantable device being tracked based on the extracted implant signal.

In some embodiments of the method for tracking an implantable device, the first ultrasonic backscatter comprises a first portion that includes an implant signal encoded by the implantable device into the first ultrasonic backscatter, and a second portion that does not include the implant signal. In some embodiments, the method comprises determining the first signal strength of the implant signal based on comparing the first portion and the second portion of the first ultrasonic backscatter.

In some embodiments of the method for tracking an implantable device, the location of the implantable device is estimated after establishing the synchronization state.

In some embodiments of the method for tracking an implantable device, the location of the implantable device is estimated based on receive beamforming.

In some embodiments of the method for tracking an implantable device, the method comprises determining a focal point associated with a local maximum signal strength comprising, iteratively: estimating the location of the implantable device; directing the ultrasonic beam from a current focal point to a test focal point based on a direction of the estimated location of the implantable device relative to the current focal point, wherein the current focal point becomes a previous focal point; determining a signal strength based on ultrasonic backscatter when the ultrasonic beam is emitted to the test focal point; and comparing the signal strength when the ultrasonic beam is emitted to the test focal point to the signal strength when the ultrasonic beam is emitted to the previous focal point. In some embodiments, the method comprises, in response to determining the focal point associated with the local maximum, establishing a steady state with the implantable device, wherein, if the signal strength decreases below a second predetermined threshold, the focal point associated with the local maximum signal is re-determined.

In some embodiments of the method for tracking an implantable device, determining whether to maintain where the emitted ultrasonic beam is being focused comprises monitoring a movement of an interrogator; and determining an adjustment to a focal point of the ultrasonic beam based on the monitored movement.

In some embodiments of the method for tracking an implantable device, the method comprises is performed at an interrogator device.

In some embodiments of a system for tracking an implantable device powered using ultrasonic waves, the system comprises: a transducer array comprising a plurality of transducers; and a controller configured to: establish a synchronization state with the implantable device, comprising: control the transducer array to emit an ultrasonic beam to a first focal point and receive a first ultrasonic backscatter corresponding to the emitted ultrasonic beam; determine a first signal strength based on the first ultrasonic backscatter; and establish the synchronization state with the implantable device in response to determining that the first signal strength is at or above a predetermined threshold; estimate a location of the implantable device; control the transducer array to emit the ultrasonic beam to a second focal point closer to the estimated location than the first focal point and receiving second ultrasonic backscatter corresponding to the emitted ultrasonic beam; determine a second signal strength based on the second ultrasonic backscatter; and determine whether to maintain or to adjust where the emitted ultrasonic beam is being focused based on comparing the determined second signal strength with the first signal strength.

In some embodiments of a method for discovering an implantable device powered using ultrasonic waves, the method comprises: emitting an ultrasonic beam to successively focus on a plurality of focal points; at each focal point of the plurality of focal points: holding the focused ultrasonic beam at the focal point for a duration that permits the implantable device, if located at the focal point, to convert energy from ultrasonic waves of the ultrasonic beam into electrical energy to enter into a powered-on state from a powered-off state, receiving an ultrasonic backscatter corresponding to the ultrasonic beam focused on the focal point, and comparing the received ultrasonic backscatter with a predetermined pattern associated with the implantable device to be discovered to generate a score indicating how likely the ultrasonic backscatter comprises the predetermined pattern; and determining a location of the implantable device from the plurality of focal points based on a plurality of scores generated for each focal point within the plurality of focal points.

In some embodiments of the method for discovering the implantable device, the method comprises causing the implantable device to enter into the powered-on state.

In some embodiments of the method for discovering the implantable device, the method further comprises establishing an ultrasonic communication link with the implantable device using ultrasonic waves emitted by the interrogator focused at the focal point corresponding to the determined location of the implantable device.

In some embodiments of the method for discovering the implantable device, the plurality of focal points corresponds to a steerable range of the ultrasonic beam.

In some embodiments of the method for discovering the implantable device, the predetermined pattern comprises one or more square waves.

In some embodiments of the method for discovering the implantable device, the predetermined pattern uniquely identifies the implantable device.

In some embodiments of the method for discovering the implantable device, the predetermined pattern comprises information encoded by the implantable device into the ultrasonic backscatter. In some embodiments, the implantable device receives the ultrasonic waves from the emitted ultrasonic beam and encodes the information into the ultrasonic backscatter by modulating an electric signal generated based on the ultrasonic waves received at the implantable device.

In some embodiments of the method for discovering the implantable device, determining the location of the implantable device comprises selecting a focal point from a subset of focal points within the plurality of focal points, wherein the score corresponding to each focal point within the subset of focal points is above a predetermined threshold value.

In some embodiments of the method for discovering the implantable device, determining the location of the implantable device comprises selecting a focal point from the plurality of focal points as being the most likely location of the implantable device based the plurality of scores.

In some embodiments of the method for discovering the implantable device, the method comprises confirming the location of the implantable device, comprising emitting the ultrasonic beam to focus on the selected focal point for a predetermined time period; and analyzing an ultrasonic backscatter received while the ultrasonic beam is focused on the selected focal point to confirm that the implantable device is located at the selected focal point. In some embodiments, the method comprises, in response to confirming that the implantable device is located at the selected focal point, maintaining the ultrasonic beam at the selected focal point.

In some embodiments, the method for discovering the implantable device is performed at an interrogator device. In some embodiments, the interrogator comprises a plurality of transducers in a transducer array, and wherein emitting the ultrasonic beam to successively focus on the plurality of focal points comprises controlling the plurality of transducers to transmit ultrasonic waves in the ultrasonic beam to successively focus on the plurality of focal points. In some embodiments, emitting the ultrasonic beam comprises successively directing the focused ultrasonic beam at each focal points of the plurality of focal points in a steerable angular range of the transducer array. In some embodiments, emitting the ultrasonic beam comprises mechanically moving the transducer array to successively direct the focused ultrasonic beam at each focal points of the plurality of focal points. In some embodiments, emitting the ultrasonic beam comprises controlling when power is supplied to each transducer in the transducer array to successively direct the focused ultrasonic beam at each focal points of the plurality of focal points.

In some embodiments of the method for discovering the implantable device, the implantable device comprises one or more capacitors to store the electrical energy converted from the ultrasonic waves of the ultrasonic beam to enter into the powered-on state from the powered-off state.

In some embodiments of the above-described methods, the ultrasonic beam has a spot size of less than 10 mm.

In some embodiments, an interrogator device for discovering an implantable device powered using ultrasonic waves, comprising: a transducer array comprising a plurality of transducers; and a controller configured to: control the transducer array to emit an ultrasonic beam successively focused on a plurality of focal point; at each focal point of the plurality of focal points: hold the focused ultrasonic beam at the focal point for a duration that permits the implantable device, if located at the focal point, to convert energy from ultrasonic waves of the ultrasonic beam into electrical energy and enter into a powered-on state from a powered-off state, receive an ultrasonic backscatter corresponding to the emitted ultrasonic beam, and compare the received ultrasonic backscatter with a predetermined pattern associated with the implantable device to be discovered to generate a score indicating how likely the ultrasonic backscatter comprise the predetermined pattern; and determine a location of the implantable device from the plurality of focal points based on a plurality of scores generated for the plurality of corresponding focal points.

Further described herein are various system embodiments for operating an implantable device using ultrasonic waves, according to any of the aforementioned method embodiments.

### BRIEF DESCRIPTION OF THE DRAWINGS

The foregoing summary, as well as the following detailed description of embodiments, is better understood when read in conjunction with the appended drawings. For the purpose of illustrating the present disclosure, the drawings show example embodiments of the disclosure; the disclosure, however, is not limited to the specific methods and instrumentalities disclosed. In the drawings:

FIG. 1 illustrates a system for powering an implantable device using ultrasonic waves emitted by an interrogator, according to some embodiments;

FIG. 2 illustrates panels showing portions of emitted ultrasonic waves for powering an implantable device, according to some embodiments;

FIG. 3 illustrate panels showing how an interrogator processes an ultrasonic backscatter received at the interrogator, according to some embodiments;

FIG. 4A illustrates an example diagram that shows how an interrogator controls where an ultrasonic (US) beam is being focused to discover and power an implantable device, according to some embodiments;

FIG. 4B illustrates an example diagram that shows how an interrogator controls where an US beam is being focused to effectively power an implantable device, according to some embodiments;

FIG. 5 illustrates an interrogator configured to power one or more implantable devices using ultrasonic waves, according to some embodiments;

FIG. 6 illustrates an implantable device that is powered and operated using ultrasonic waves, according to some embodiments;

FIG. 7 illustrates a method for discovery an implantable device using ultrasonic waves, according to some embodiments;

FIG. 8 illustrates a diagram showing example operating logic of an interrogator to effectively power an implantable device using ultrasonic waves, according to some embodiments;

FIG. 9 illustrates a method for tracking an implantable device powered using ultrasonic waves, according to some embodiments;

FIG. 10 illustrates a method for tracking an implantable device powered using ultrasonic waves to effectively maintain power supplied to the implantable device, according to some embodiments;

FIG. 11 illustrates a method for tracking an implantable device powered using ultrasonic waves to effectively maintain power supplied to the implantable device, according to some embodiments;

FIG. 12 illustrates an example diagram showing a pattern encoded by an implantable device into an ultrasonic backscatter received by an interrogator, according to some embodiments;

FIG. 13 illustrates example charts that show how accurately an interrogator estimates a location of an implantable device in a discovery mode, according to some embodiments; and

FIG. 14 illustrates a diagram of an implantable device configured to interact with a nerve of a subject, according to some embodiments.

### DETAILED DESCRIPTION

Described herein are systems and methods for discovering and tracking the device implantable within a subject using ultrasonic waves emitted by an interrogator. The implantable device can include an ultrasonic transducer configured to receive ultrasonic waves emitted by an interrogator and convert the mechanical energy of the received ultrasonic waves into an electrical energy. Because the implantable device receives power transmitted by ultrasonic waves, power transfer from the interrogator should be efficient and reliable. In some embodiments, to provide these functionalities, the interrogator needs to be capable of communicating with the implantable device to assess whether power is being efficiently conveyed by the emitted ultrasonic waves to the implantable device. In some embodiments, the implantable device can be configured to modulate an electrical signal at an ultrasonic transducer on the implantable device to embed an implant signal within an ultrasonic backscatter corresponding to ultrasonic waves emitted by the interrogator. For example, the embedded signal may include information generated by or is associated with the implantable device.

Through this mechanism, the interrogator can be configured to derive a signal strength of an implant signal extracted from received ultrasonic backscatter and use the derived signal strength as an indication for how effectively ultrasonic power is being conveyed to the implantable device. For example, due to misalignment between the interrogator's emitted ultrasonic (US) beam and the implantable device, which may be caused by patient or interrogator operator motion, the derived signal strength will be low or decrease. Accordingly, the interrogator can be configured to control a beam focus of an US beam to increase the alignment and therefore maximize power incident on an ultrasonic transducer of the implantable device. Moreover, the interrogator can be configured to monitor the signal strength determined from ultrasonic backscatter to track the implantable device as its position shifts to maintain alignment with and efficient power deliver to the implantable device.

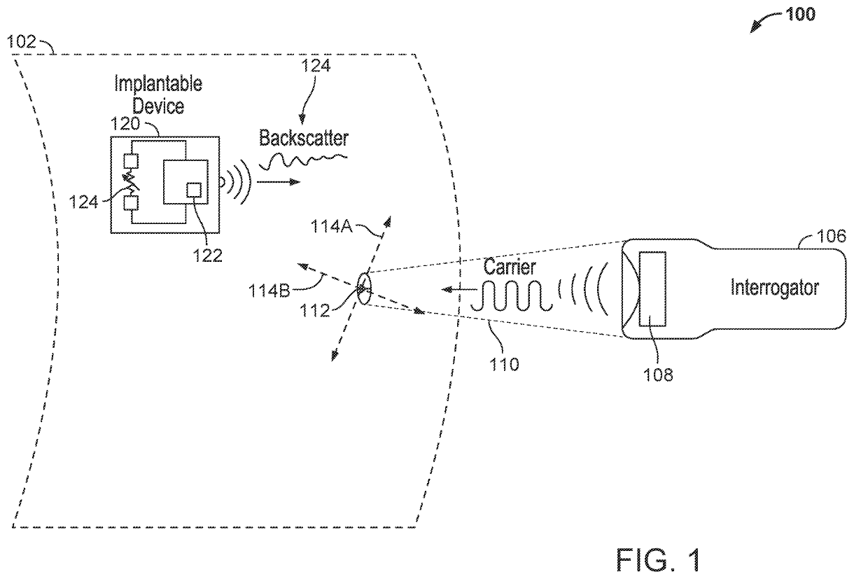

FIG. 1 illustrates a system 100 for powering an implantable device 120 using ultrasonic waves emitted by an interrogator 106, according to some embodiments. In some embodiments, implantable device 120 can be implanted within a subject such as a patient and interrogator 106 can be a separate device that is external to (i.e., non-implanted) or fully-implanted in the subject. As shown in system 100, implantable device 120 can be located in a region 102 (e.g., an area of skin of the subject) and implanted within the subject.

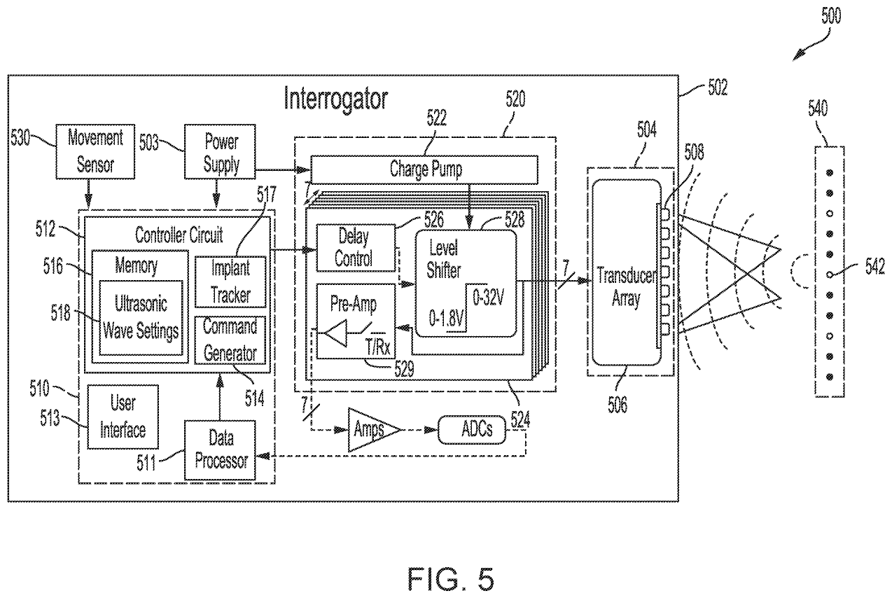

In some embodiments, interrogator 106 can be configured to control a plurality of ultrasonic transducers 108 to emit ultrasonic waves narrowed into an ultrasonic (US) beam 110 to power implantable device 120. For example, as will be further described below with respect to FIG. 5, ultrasonic transducers 108 can be provided as a transducer array and interrogator 106 can individually control the ultrasonic transducers 108 to generate US beam 110 in a technique known as electronic beam forming. As a result of this technique, the wave fronts of ultrasonic waves emitted by the plurality of ultrasonic transducers 108 will intersect at a focal point 112, which corresponds to a specific portion of US beam 110 with the highest beam intensity. Focal point 112 also corresponds to the narrowest part of the beam diameter of US beam 110. Therefore, interrogator 106 may convey ultrasonic power of US beam 110 to a limited region, i.e., focal point 112. Further, interrogator 106 can be configured to individually control ultrasonic transducers 108 to change a position of focal point 112. In some embodiments, interrogator 106 can generate US beam 110 having a spot size of about 1 mm or less, about 2 mm or less, about 3 mm or less, about 5 mm or less, about 7 mm or less, or about 10 mm or less. In some embodiments, interrogator 106 can generate US beam 110 having a spot size of at least 0.5 mm, at least 1 mm, at least 2 mm, at least 3 mm, at least 5 mm, or at least 7 mm. In some embodiments, the spot size can be between around 2-8 mm, 2-5 mm, or 2-4 mm.

In some embodiments where ultrasonic transducers 108 are elements of a 2D transducer array, interrogator 106 can change a position of focal point 112 of US beam 110 within a plane as represented by the perpendicular axes 114A and 114B. In other words, interrogator 106 may direct focal point 112 to a plurality of positions within a steerable range of ultrasonic transducers 108, which may encompass, for example, region 102. In some embodiments, as will be further described below, interrogator 106 can control where US beam 110 is being focused to increase alignment between US beam 110 and implantable device 120. Increased alignment not only enables ultrasonic power to be more effectively conveyed to implantable device 120, but also increases higher device reliability and safety since ultrasonic power does not need to be increased beyond unsafe levels. As shown, interrogator 106 may transmit ultrasonic waves comprising a carrier signal in the form of US beam 110.

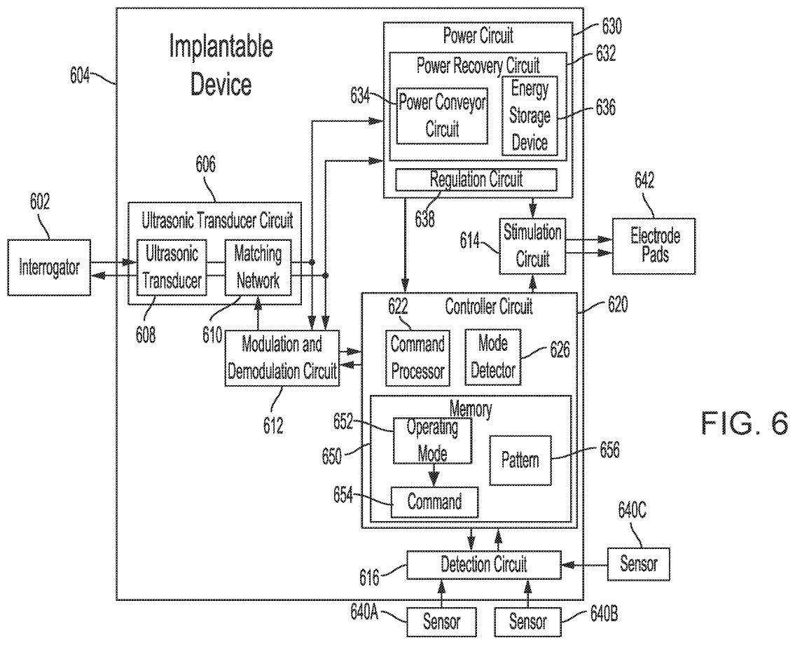

In some embodiments, implantable device 120 can be wirelessly powered and operated by ultrasonic waves emitted from interrogator 106, as will be further described below with respect to FIGS. 5-6. For example, implantable device 120 can include one or more ultrasonic transducers 122 configured to receive ultrasonic waves of US beam 110 and convert mechanical energy of the ultrasonic waves into electrical energy to power and operate implantable device 120. For example, implantable device 120 may include one or more sensors 124 that can be controlled to detect or measure a physiological condition of the patient. The better aligned that US beam 110 is with implantable device 120, i.e., focal point 112 is closer to implantable device 120, the more mechanical energy the one or more ultrasonic transducers 122 of implantable device 120 can extract from ultrasonic waves of US beam 110.

In some embodiments, to enable interrogator 106 to track or discover implantable device 120 using ultrasonic waves, implantable device 120 can be configured to wirelessly communicate with interrogator 106 through ultrasonic communication. In particular and as will be further described below with respect to FIG. 6, implantable device 120 can be configured to modulate an electrical signal of ultrasonic transducer 122 to embed an implant signal within an ultrasonic backscatter 124. In some embodiments, the implant signal can include information derived or generated by implantable device 120. For example, implantable device 120 may embed information including measurements generated by sensors 124. In other embodiments, the implant signal can include a predetermined pattern associated with implantable device 120.

In some embodiments, interrogator 106 can be configured to switch between a transmit mode and a receive mode to emit and receive ultrasonic waves, respectively. In the transmit mode, interrogator 106 can emit US beam 110. In the receive mode, interrogator 106 may be configured to receive and analyze ultrasonic backscatter 124. In some embodiments, as will be further described below, interrogator 106 can extract an implant signal from received ultrasonic backscatter 124 to determine whether and how to adjust a position of focal point 112 to increase alignment between US beam 110 and implantable device 120. For example, interrogator 106 may determine and monitor a signal strength of the extracted implant signal to determine how to adjust the position of focal point 112. In some embodiments, interrogator 106 can receive ultrasonic backscatter 124 through receive beamforming. Based on received ultrasonic backscatter 124, interrogator 106 can estimate a location of implantable device 102 and direct focal point 112 in a direction towards the estimated location.

In some embodiments, interrogator 106 can be configured to discover implantable device 120 by analyzing whether an implant signal is received in ultrasonic backscatter 124. For example, implantable device 120 may be initially in a powered-off state. In some embodiments, interrogator 106 can be configured to sweep its US beam 110 across a plurality of focal points in region 102 to supply enough ultrasonic power to cause implantable device 120 to change from the powered-off state to a powered-on state. In some embodiments, during a start-up phase, implantable device 120 can be configured to embed, within ultrasonic backscatter 124, an implant signal identifying implantable device 120. In some embodiments, interrogator 106 can assess how likely the implant signal is present in received ultrasonic backscatter at the plurality of focal points to estimate the location and therefore discover the initially powered-off implantable device.

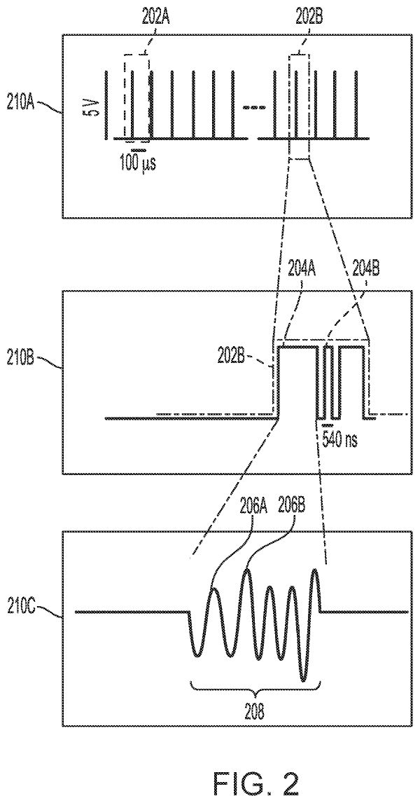

FIG. 2 illustrates panel 210A-C showing portions of emitted ultrasonic waves for powering an implantable device, according to some embodiments. For example, the ultrasonic waves shown in panels 210A-C may be emitted by interrogator 106 of FIG. 1 (or interrogator 502 of FIG. 5) within US beam 110.

Panel 210A shows that the emitted ultrasonic waves include a series of ultrasonic wave commands such as ultrasonic wave commands 202A and 202B. In some embodiments, an ultrasonic wave command may be received and decoded by an implantable device receiving the ultrasonic waves to control operations of the implantable device. For example, an ultrasonic wave commands may include a command to power the implantable device from a powered-off state to a powered-on state. Other example ultrasonic wave commands may include a command to request the implantable device to detect a physiological condition of the subject and/or to transmit the detected condition back to the interrogator via an emitted ultrasonic backscatter.

In some embodiments, each of the ultrasonic wave commands may include a predetermined pattern of one or more pulses of ultrasonic waves (i.e., also known as ultrasound pulses). For example, panel 210B shows a zoomed-in view of ultrasonic wave command 202B, which may include a sequence of three ultrasound pulses (e.g., pulses 204A-B). For illustration purposes only, the amplitude (i.e., pressure amplitude) and pulse width (i.e., also called pulse length or pulse duration) of each pulse in ultrasonic wave command 202B is shown as being different, but, this may not be the case. In some embodiments, the amplitude or pulse width of each ultrasound pulse may be dictated by an ultrasonic wave protocol implemented by the interrogator. Therefore, the amplitudes and pulse width of the pulses may be the same or different depending on the ultrasonic wave protocol. In some embodiments, each unique ultrasonic wave command may include a predetermined pattern uniquely identifying the ultrasonic wave command. The predetermined pattern may comprise a number of pulses each having specific characteristics (e.g., amplitude and pulse width).

In some embodiments, each of the ultrasound pulses may include one or more carrier cycles (i.e., also known as vibration or oscillation cycles or carrier waves). As used in the present disclosure herein, a carrier cycle may correspond to a single oscillation of the ultrasonic waves. For example, panel 210C shows a zoomed-in view of ultrasound pulse 204A that includes five carrier cycles (e.g., ultrasound cycles 206A-B) that comprise a pulse duration 208 of ultrasound pulse 204A. In some embodiments, a single ultrasound pulse may include a wave pattern comprising a plurality of carrier cycles to encode specific information such as a specific ultrasonic wave command. For example, the wave pattern may include a plurality of carrier cycles in which at least two carrier cycles have different wavelengths or different amplitudes. As discussed above, the signal characteristics of the plurality of carrier cycles within ultrasound pulse 204A may be dictated by the ultrasonic wave protocol to represent specific ultrasound wave commands. In some embodiments, by permitting the carrier cycles of ultrasound pulse 204A to be non-uniform, more types of ultrasonic wave commands can be encoded to communicate with implantable devices.

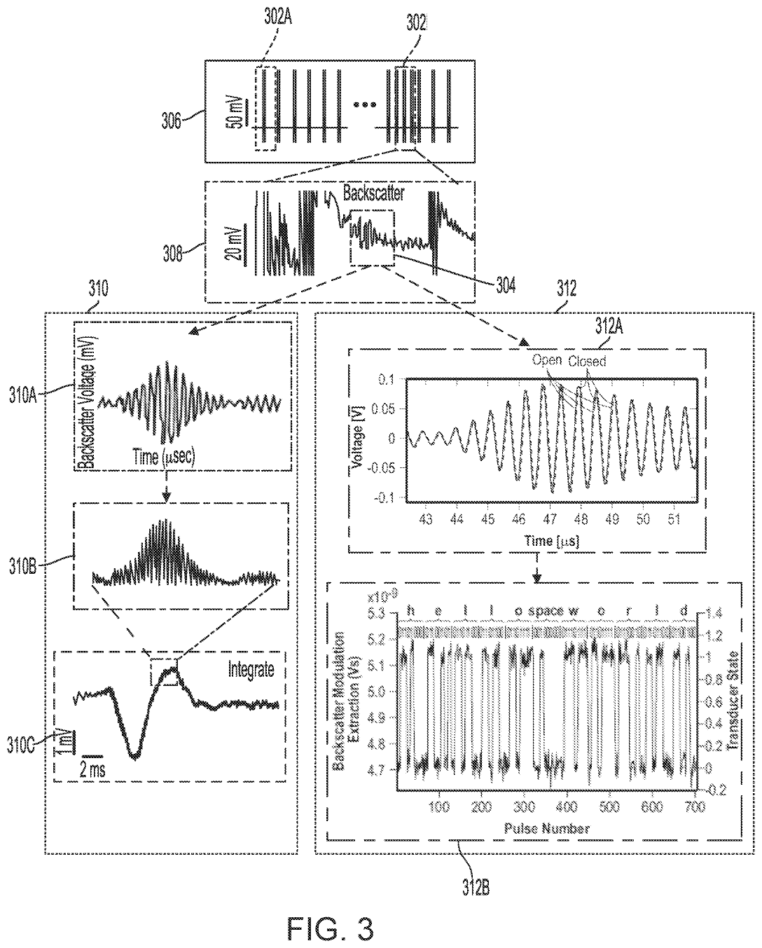

FIG. 3 illustrates panels showing how an interrogator (e.g., interrogator 106) processes an ultrasonic backscatter received at the interrogator, according to some embodiments. In some embodiments, an implantable device (e.g., implantable device 120 of FIG. 1 or implantable device 602 of FIG. 6) can be configured to emit the ultrasonic backscatter, as shown in panel 306, in response to receiving ultrasonic waves such as those described above with respect to panel 210A of FIG. 2. As described above with respect to FIG. 1, the implantable device can be configured to modulate an electrical signal of one or more of its transducers to encode implant data within the emitted ultrasonic backscatter. As will be further described below with respect to FIG. 6, the implant data may include a response to an ultrasonic wave command. For example, the implant data may include sensor data measured at the implantable device. In another example, the implant data may include a unique identifier of the implantable device (e.g., a serial number).

Panel 306 shows the ultrasonic backscatter received at the interrogator from the implantable device. In some embodiments, the ultrasonic backscatter can correspond to a backscatter of the ultrasonic waves transmitted to the implantable device, as shown in panel 210A of FIG. 2. As shown in panel 306, the ultrasonic backscatter can include backscattered portions 302A-B that correspond to a backscatter of the operating mode command portions of the transmitted ultrasonic waves of panel 210A. In some embodiments, at the end of a transmit cycle, the interrogator can be configured to control a switch (e.g., switch 529 of FIG. 5) to disconnect the transmit module and connect the receive module to receive the ultrasonic backscatter.

Panel 308 shows a zoomed-in view of a backscatter of a single ultrasonic pulse 304, which can be analyzed to extract data encoded by the implantable device into the backscatter 304. In some embodiments, backscatter 304 can be analyzed through analog signal processing 310. In some embodiments, backscatter 304 can be analyzed through digital signal processing 312.

In some embodiments, analog signal processing 310 include a series of steps shown in panels 310A-C. For example, as shown in panel 310A, the ultrasonic backscatter 304 can be filtered. In some embodiments, the ultrasonic waves transmitted by the interrogator are reflected off of the implantable device such as a surface of an ultrasonic transducer of the implantable device. The amplitude of the backscatter waves reflected from the surface of the transducer can change as a function of changes in impedance of the current returning to the ultrasonic transducer, and can be referred to as the “responsive backscatter” since this backscatter encodes information generated at the implantable device. For example, the amplitude characteristics of portions of the ultrasonic backscatter shown in panel 310A may depend on how the implantable device modulates the electrical signal of the ultrasonic transducer. These changes may enable the interrogator to better align the US beam with the implantable device to increase power efficiencies as well as ultrasonic communication reliability, as will be further described below. Further analysis of the filtered backscatter may include rectifying the ultrasonic backscatter, as shown in panel 310B, and integrating the rectified signal to decode data, as shown in panel 310C.

In some embodiments, digital signal processing 312 include a series of steps shown in panels 312A-B. Similar to panel 310A, panel 312A shows a zoomed-in view of a filtered backscatter 304. As described above with respect to FIG. 1 and will be further described below with respect to FIG. 6, the implantable device can modulate its acoustic impedance by shunting its piezoelectric ultrasonic transducer across a digitally controlled switch where a high level corresponds to an open configuration and a low level corresponds to a closed configuration.

Panel 312A shows the difference in amplitude of the filtered signals of backscatter 304 depending on whether the implantable device's transducer is in the shorted/closed or opened configuration. In some embodiments, the implantable device can control the electrodes of the ultrasonic transducer to be in the shorted and opened configurations to embed implant data within the backscatter. The change in impedance due to the switch activity results in a backscatter peak amplitude that is 11.5 mV greater in the open switch configuration compared to the closed switch configuration-a modulation depth of 6.45%.

In some embodiments, the implantable device can be configured to implement a line code to control the ultrasonic transducer switch activity to embed digital data. For example, the line code may include unipolar, polar, bipolar, or a Manchester code. The interrogator can be configured with the capability to decode the line code used by the implantable device to decode the digital data. For example, panel 312B shows modulated values on the transducer and the corresponding extracted modulation values of the transducer of the implantable device. The absolute value and noise margin of the extracted signal values depend on a variety of factors such as implantable device distance, orientation, and size, however, the extracted waveform remains representative of the modulated signal of the implantable device, varying by a linear scaling factor. For example, the implantable device may implement a pulse-amplitude-modulated non-return to zero level coding, through which an 11-character ASCII message (“hello world”) may be communicated to the interrogator. In particular, as shown in panel 312B, the interrogator can differentiate between the two transducer states of close or open configurations based on the extracted backscatter modulation voltages. These extracted transducer states may be mapped to binary values of 0 and 1 to encode digital data. In some embodiments, digital signal processing 312 can be advantageous over the analog signal processing 310 approach because the line coding protocols implemented by the implantable device may increase ultrasonic communication reliability between the implantable device and the interrogator.

In some embodiments, the information communicated by the implantable device and embedded within the emitted ultrasonic backscatter can include various data, which may be digitalized. In some embodiments, the information can include data collected or generated by the implantable device. For example, the information may include sensor data such as temperature, pressure, pH, strain, a presence of or an amount of an analyte or an electrical physiological signal such as a never action potential.

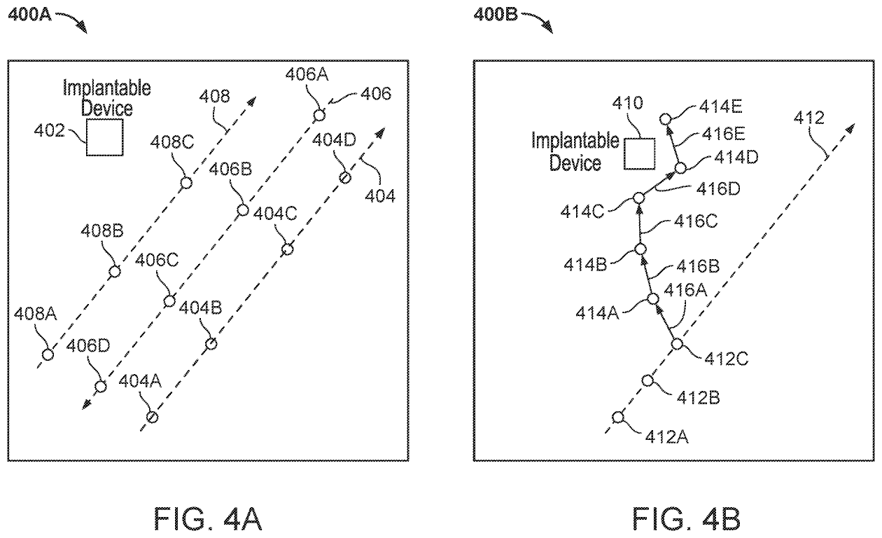

FIG. 4A illustrates an example diagram 400A that shows how an interrogator (e.g., interrogator 106 of FIG. 1) controls where an ultrasonic (US) beam is being focused to discover and power an implantable device 402, according to some embodiments. For example, diagram 400A shows a region (e.g., region 102) in which implantable device 402 is implanted with a subject or patient.

In some embodiments, in the discovery mode, the interrogator can be configured to direct the US beam to focus on a plurality of focal points 404A-D in range 404. For example, the interrogator may sweep the US beam in a linear direction from focal point 404A towards focal point 404D. In some embodiments, the interrogator can hold the US beam at each focal point for a duration that permits implantable device 402, if located within a threshold distance of the focal point, to power on from a powered-off state.

In some embodiments, the interrogator can be configured to sweep the US beam in multiple ranges including 406 and 408. For example, in each range, the interrogator may successively direct the US beam to focus on a plurality of focal points (e.g., focal points 406A-406D) in a linear direction as shown in range 406.

In some embodiments, once implantable device 402 receives enough energy from the US beam, implantable device 402 can be configured to embed a signal including a predetermined pattern within an emitted ultrasonic backscatter to broadcast its presence. For example, the predetermined pattern may be associated with implantable device 402 and may uniquely identify implantable device, according to some embodiments.

Depending on the distance between implantable device 402 and the focal point of the US beam, the signal strength of the embedded signal as received by the interrogator will vary. If the distance is too large, the embedded signal may not by easily differentiated from noise. In some embodiments, the interrogator can be configured to examine ultrasonic backscatter received for each of focal points 404A-404D, 406A-406D, and 408A-408C to determine a likelihood that the predetermined pattern associated with implantable device 402 is found in each of the ultrasonic backscatters. Then, the interrogator can be configured to statistically determine a likely location of implantable device, as will be further described below.

For example, the interrogator may determine that the predetermined pattern is most likely present in the ultrasonic backscatters received for focal points 404B and 404C. Based on this determination, the interrogator may estimate the location of implantable device 402 to be close to focal points 404B and 404C.

FIG. 4B illustrates an example diagram 400B that shows how an interrogator (e.g., interrogator 106 of FIG. 1) controls where an US beam is being focused to effectively track an implantable device 410, according to some embodiments. For example, diagram 400B shows a region (e.g., region 102) in which implantable device 411 is implanted within a subject or patient.

In some embodiments, the interrogator may increment a position of a beam focus of an emitted US beam in a linear direction 412. For example, the interrogator may successively direct the US beam to focus on a plurality of focal points 412A-C within range 412. At each of focal points 412A-C, the interrogator may receive corresponding ultrasonic backscatter. As described above, implantable device 410 may be configured to encode, within the ultrasonic backscatter, an implant signal associated with implantable device 410. For example, the implant signal may be a predetermined pattern associated with implantable device 110. In some embodiments, the interrogator can be configured to extract the implant signal from the ultrasonic backscatter and determine a signal strength of the extracted signal.

In some embodiments, the signal strength represents a signal-to-noise ratio determined from the ultrasonic backscatter. In some embodiments, at each focal point, the interrogator can be configured to transmit multiple ultrasound pulses and the implantable device may be configured to encode information in ultrasonic backscatter corresponding to a portion of those ultrasound pulses. Accordingly, the interrogator can compare the extracted signal with ultrasonic backscatter that does not include the extracted signal to determine the signal strength. In some embodiments, the implantable device can be configured to toggle between a passive mode in which no signal modulation occurs and an active mode in which modulation occurs. In both embodiments, the interrogator can be configured to compare a first backscattered signal corresponding to no signal modulation with a second backscattered signal corresponding to signal modulation to cancel environmental interference or noise. For example, the interrogator may be configured to subtract the first backscattered signal (i.e., passive reflectance where no modulation occurs) from the second backscattered signal such that environmental noise can be canceled.

In some embodiments, the interrogator can be configured to determine the signal strength of the filtered backscatter signal by determining a modulation depth or an amplitude variation of the backscatter signal. For example, the interrogator may determine the percentage of amplitude variation of the backscattered signal to determine the signal strength.

In some embodiments, once the interrogator determines that the signal strength for a focal point, e.g., focal point 412C, exceeds a predetermined threshold, the interrogator determines that the focal point is within a “close” distance of implantable device 410. Accordingly, the interrogator can enter a signal optimization state in which the interrogator incrementally adjusts a position of the beam focus to approach a location of implantable device 410.

In some embodiments, the interrogator can estimate a location of implantable device 410 based on receive beam forming. Based on this location, the interrogator can increment the position of focal point 412C towards direction 416A at focal point 414A. Thereafter, the interrogator can similarly determine a signal strength of ultrasonic backscatter received at the updated focal point to determine if the signal strength is increasing, i.e., higher than that determined at the previous focal point. Accordingly, the interrogator can incrementally adjust the focal point from focal point 414A to 414E in respective directions 416B-414E until the interrogator determines that an extracted signal strength is no longer increasing. At this point, the interrogator may determine that focal point 414E is closely aligned to the true location of implantable device 410 since an extracted signal strength is at a local maximum.

In some embodiments, once this focal point 414E is determined, the interrogator can be configured to maintain the beam focus of the US beam at focal point 414E until implantable device 410 becomes misaligned from the interrogator. For example, due to movement of an operator of the interrogator and movement of a subject in which implantable device 410 is implanted, the distance between implantable device 410 and focal point 414E may exceed a threshold distance representing an acceptable distance. In some embodiments, the interrogator can determined whether such a misalignment occurs my monitoring the signal strength extracted from ultrasonic backscatter while the US beam is targeted at focal point 414E. In some embodiments, the interrogator can re-enter a tracking mode to adjust the beam focus once the misalignment has been detected.

FIG. 5 illustrates a system 500 including an interrogator 502 configured to power one or more implantable devices 540 using ultrasonic waves, according to some embodiments. In some embodiments, interrogator 502 can be an example of interrogator 106 as described above with respect to FIG. 1.

In some embodiments, interrogator 502 includes a power supply 503, a computational circuit 510, a signal-generation circuit 520, and an ultrasonic transducer circuit 504. As shown, power supply 503 can be configured to power computational circuit 510 and signal-generation circuit 520. In some embodiments, power supply 503 can provide 1.8V, although any suitable voltage can be used. For example, power supply 503 may include one or more batteries to supply the 1.8V.

In some embodiments, signal-generation circuit 520 includes a charge pump 522 configured to power one or more channels 524. In some embodiments, charge pump 522 can be configured to increase the voltage provided by power supply 503. For example, charge pump 522 may increase the 1.8V supplied by power supply 503 to 32V. In some embodiments, as will be further described below, signal-generation circuit 520 can individually power and control each ultrasonic transducer 508 of transducer array 504 to generate and emit an US beam whose ultrasonic waves are narrowed to a focal point (e.g., focal point 112 of US beam 110 shown in FIG. 1.)

In some embodiments, each channel 524 is coupled to and controls an operation of a corresponding ultrasonic transducer 508 of transducer circuit 504. In some embodiments, ultrasonic transducer 508 connected to channel 524 can be configured only to receive or only to transmit ultrasonic waves, in which case switch 529 can be optionally omitted from channel 524. In some embodiments, each channel 524 can include the following electronic components: a delay control 526, a level shifter 528, and a switch 529.

In some embodiments, delay control 526 can be configured to control the waveforms and/or signals of ultrasonic waves transmitted by ultrasonic transducer 508. In some embodiments, delay control 526 can control, for example, a phase shift, a time delay, a pulse frequency, a wave shape (including amplitude and wavelength), or a combination thereof based on commands from controller circuit 512 to generate the transmit waveform. In some embodiments, the data representing the wave shape and frequency for each channel can be stored in a ‘wave table’ stored in delay control 526 or in memory 516. This may allow the transmit waveform on each channel 524 to be different.

In some embodiments, delay control 526 can be connected to a level shifter 528 that is configured to shift input pulses from delay control 526 to a higher voltage used by ultrasonic transducer 508 to transmit the ultrasonic waves. In some embodiments, delay control 526 and level shifter 528 can be configured to be used to stream data to the actual transmit signals to transducer array 506. In some embodiments, transducer array 506 can be a linear array of ultrasonic transducers. In other embodiments, transducer array 506 can be a 2D array of ultrasonic transducers. In some embodiments, transducer array 506 can include a phased-array of linear ultrasonic transducers. In other embodiments, transducer array 506 can include a linear curved array or a curvilinear array of ultrasonic transducers. In some embodiments, the transmit waveform for each channel 524 can be produced directly by a high-speed serial output of a microcontroller or other digital system and sent to the transducer element (e.g., ultrasonic transducer 508) through level shifter 528 or a high-voltage amplifier.

In some embodiments, switch 529 of channel 524 can configure a corresponding ultrasonic transducer 508 to receive ultrasonic waves such as an ultrasonic backscatter. In some embodiments, the received ultrasonic waves are converted to an electrical current by ultrasonic transducer 508 (set in a receiving mode) and transmitted to data processor 511 to process data captured in the received ultrasonic waves. For example, data processor 511 can be configured to implement receive beam forming to enable interrogator 502 to estimate and determine a location of implantable devices 540. In some embodiments, an amplifier, an analog-to-digital converter (ADC), a variable-gain-amplifier, or a time-gain-controlled variable-gain-amplifier which compensates for tissue loss, and/or a band pass filter can be included to process the received ultrasonic waves.

In some embodiments, channel 524 described above does not include a T/Rx switch 529, but instead contains independent Tx (transmit) and Rx (receive) with a high-voltage Rx (receiver circuit) in the form of a low noise amplifier with good saturation recovery. In some embodiments, the T/Rx circuit includes a circulator. In some embodiments, transducer array 506 includes more transducer elements (e.g., ultrasonic transducer 508) than processing channels 524, and interrogator 502 can be configured to include a multiplexer to select different sets of transmitting elements for each pulse. For example, 64 transmit/receive channels may be connected via a 3:1 multiplexer to 192 physical transducer elements—with only 64 transducer elements active on a given pulse.

In some embodiments, interrogator 502 can include a movement sensor 530, which may include one or more movement sensors. In some embodiments, movement sensor 530 can be configured to detect and measure a movement of interrogator 502. For example, interrogator 502 may move due to a movement or a hand jitter of an operator of interrogator 502. In some embodiments, movement sensor 530 can include one or more of an accelerometer, a gyroscope, or an inertial movement unit (EMU).

In some embodiments, computational circuit 510 can be a digital circuit, an analog circuit, or a mixed-signal integrated circuit. Examples of computational circuit 510 may include a microprocessor, a finite state machine (FSM), a field programmable gate array (FPGA), and a microcontroller. In some embodiments, interrogator 502 can include a volatile memory, which can be accessed by computational circuit 510.

In some embodiments, computational circuit 510 includes controller circuit 512, data processor 511, and user interface 513. In some embodiments, controller circuit 512 includes command generator 514, implant tracker 517, and memory 516 storing ultrasonic wave settings 518.

In some embodiments, command generator 514 can be configured to generate instructions to control operation of delay control 526 to transmit one or more operating mode commands to one or more implantable devices 540 to operate the one or more implantable devices 540. For example, the operating mode command can instruct an implantable device (e.g., implantable device 542) receiving the operating mode command to upload certain device data or to download data encoded in the operating mode command.

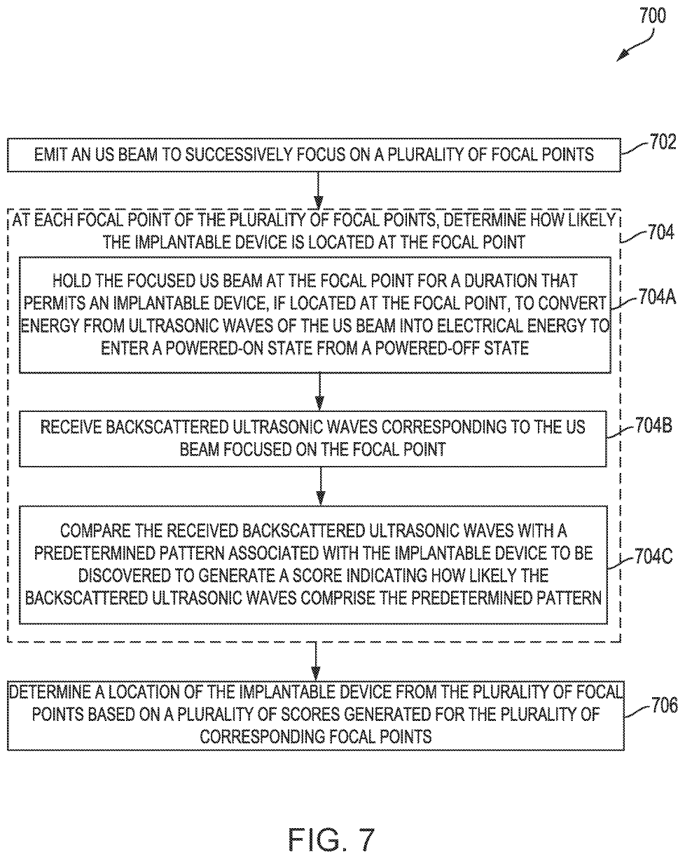

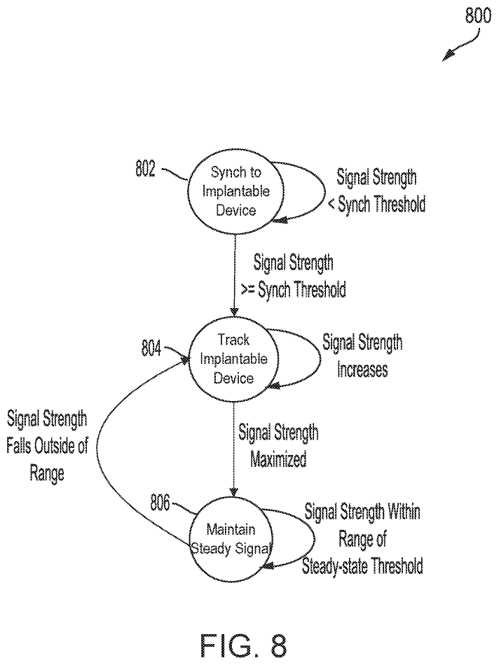

In some embodiments, implant tracker 517 can be configured to operate in a plurality of modes to track implantable devices 540. In some embodiments, implant tracker 517 can operate in a discovery mode to detect an initially powered-off implantable device 542, as will be further described below with respect to FIG. 7. In some embodiments, implant tracker 517 can operate in a tracking mode to track a location of implantable device 542, as will be further described below with respect to FIGS. 8-11. In some embodiments, implant tracker 517 can be configured to analyze movement data generated by movement sensor 530 to determine whether and how to adjust the beam focus of US beam to counteract the movement of interrogator 502 caused by the operator. In both modes, implant tracker 517 can be configured to control ultrasonic transducer circuit 504 to change a focal point of an emitted US beam.

In some embodiments, the device data received and processed by data processor 511 can include information embedded by implantable device 542 within received ultrasonic backscatter. In these embodiments, command generator 514 can be configured to set or select ultrasonic wave settings to control ultrasonic transducers of transducer array 504 to change or maintain a focal point of an emitted US beam.

In some embodiments, transducer circuit 504 includes one or more ultrasonic transducers 508 configured to transmit ultrasonic waves to power implantable devices 540 such as implantable device 542. In some embodiments, as shown in FIG. 5, transducer circuit 504 includes transducer array 506 having a plurality of ultrasonic transducers 508. In some embodiments, transducer array 506 includes 1 or more, 2 or more, 3 or more, 5 or more, 7 or more, 10 or more, 15 or more, 20 or more, 25 or more, 50 or more, 100 or more 250 or more, 500 or more, 1000 or more, 2500 or more, 5000 or more, or 10,000 or more ultrasonic transducers. In some embodiments, transducer array 206 includes 100,000 or fewer, 50,000 or fewer, 25,000 or fewer, 10,000 or fewer, 5000 or fewer, 2500 or fewer, 1000 or fewer, 500 or fewer, 200 or fewer, 150 or fewer, 100 or fewer, 90 or fewer, 80 or fewer, 70 or fewer, 60 or fewer, 50 or fewer, 40 or fewer, 30 or fewer, 25 or fewer, 20 or fewer, 15 or fewer, 10 or fewer, 7 or fewer or 5 or fewer ultrasonic transducers. Transducer array 506 may be, for example, a chip comprising 50 or more ultrasonic transducer pixels.

As shown in FIG. 5, transducer circuit 504 includes a single transducer array 506; transducer circuit 504, however, can include 1 or more, 2 or more, or 3 or more separate transducer arrays, according to some embodiments. In some embodiments, transducer circuit 504 includes 10 or fewer transducer arrays (such as 9, 8, 7, 6, 5, 4, 3, 2, or 1 transducer arrays). In some embodiments, the separate transducer arrays can be placed at different points of a subject, and can communicate to the same or different implantable devices 540. In some embodiments, the transducer arrays can be located on opposite sides of an implantable device such as implantable device 542.

In some embodiments, the specific design of transducer array 506 of interrogator 502 depends on the desired penetration depth, aperture size, and size of the individual ultrasonic transducers 508 within transducer array 506. The Rayleigh distance, R, of the transducer array 506 is computed as:

$$R = \frac{{D}^{2} - {λ}^{2}}{4 λ} \approx \frac{{D}^{2}}{4 λ} , {D}^{2} ≫ {λ}^{2}$$

where D is the size of the aperture and X is the wavelength of ultrasound in the propagation medium (i.e., the tissue). As understood in the art, the Rayleigh distance is the distance at which the beam radiated by transducer array 506 is fully formed. That is, the pressure filed converges to a natural focus at the Rayleigh distance to maximize the received power. Therefore, in some embodiments, implantable devices 540 can be approximately the same distance from transducer array 506 as the Rayleigh distance.

The individual ultrasonic transducers 508 in transducer array 506 can be modulated to control the Raleigh distance and the position of the beam of ultrasonic waves emitted by transducer array 506 through a process of beamforming or beam steering. Techniques such as linearly constrained minimum variance (LCMV) beamforming can be used to communicate a plurality of implantable devices 540 (e.g., implantable device 542) with an external ultrasonic transceiver. See, for example, Bertrand et al., Beamforming Approaches for Untethered, Ultrasonic Neural Dust Motes for Cortical Recording: a Simulation Study, IEEE EMBC (August 2014). In some embodiments, beam steering is performed by adjusting the power or phase of the ultrasonic waves emitted by ultrasonic transducers 508 in transducer array 506.

In some embodiments, interrogator 502 (e.g., computational circuit 510) includes one or more of instructions for beam steering ultrasonic waves using one or more ultrasonic transducers 508, instructions for determining the relative location of one or more implantable devices 540, instructions for monitoring the relative movement of one or more implantable devices 540, instructions for recording the relative movement of one or more implantable devices 540, and instructions for deconvoluting backscatter from a plurality of implantable devices 540.

In some embodiments, user interface 513 can be configured to allow a user (e.g., a physician or a patient) to control the operations of interrogator 502 to power or operate implantable devices 540 or to communicate with implantable devices 540. In some embodiments, user interface 513 can include an input device that provides input, such as a touch screen or monitor, keyboard, mouse, or voice-recognition device to interrogator 502. In some embodiments, user interface 513 can include an output device such as any suitable device that provides output, such as a touch screen, monitor, printer, disk drive, or speaker.

In some embodiments, interrogator 502 can be controlled using a separate computer system (not shown), such as a mobile device (e.g., a smartphone or a tablet). The computer system can wirelessly communicate to interrogator 502, for example through a network connection, a radiofrequency (RF) connection, or Bluetooth. The computer system may, for example, turn on or off interrogator 502 or analyze information encoded in ultrasonic waves received by interrogator 502.

In some embodiments, interrogator 502 communicates with a plurality of implantable devices 540. This can be performed, for example, using multiple-input, multiple output (MIMO) system theory. For example, communication between interrogator 502 and the plurality of implantable devices 540 may be performed using time division multiplexing, spatial multiplexing, or frequency multiplexing. Interrogator 502 can receive a combined ultrasonic backscatter from the plurality of the implantable devices 540, which can be deconvoluted, thereby extracting information from each implantable device 542. In some embodiments, interrogator 502 can be configured to focus the ultrasonic waves transmitted from transducer array 506 to a particular implantable device through beam steering. For example, interrogator 502 may focus the transmitted ultrasonic waves to a first implantable device (e.g., implantable device 542), receives backscatter from the first implantable device, focuses transmitted ultrasonic waves to a second implantable device, and receives backscatter from the second implantable device. In some embodiments, interrogator 502 transmits ultrasonic waves to a plurality of implantable devices 540, and then receives ultrasonic backscatter from the plurality of implantable devices 540.

In some embodiments, interrogator 502 or one or more of ultrasonic transducers 508 are wearable. For example, interrogator 502 or one or more of ultrasonic transducers 508 may be fixed to the subject's body by a strap or adhesive. In another example, interrogator 502 can be a wand, which may be held by a user (such as a healthcare professional). In some embodiments, interrogator 502 can be held to the body via suture, simple surface tension, a clothing-based fixation device such as a cloth wrap, a sleeve, an elastic band, or by sub-cutaneous fixation. In some embodiments, one or more ultrasonic transducers 508 or transducer array 506 of interrogator 502 may be positioned separately from the rest of interrogator 502. For example, transducer array 206 may be fixed to the skin of a subject at a first location (such as proximal to one or more implanted devices), and the rest of interrogator 502 may be located at a second location, with a wire tethering ultrasonic transducer 508 or transducer array 506 to the rest of interrogator 502.

FIG. 6 illustrates an implantable device 604 that is powered and operated using ultrasonic waves, according to some embodiments. In some embodiments, implantable device 604 can be wirelessly powered and operating by ultrasonic waves transmitted from interrogator 602, as described above with respect to FIG. 5. In some embodiments, implantable device 604 can be configured to wirelessly communicate with interrogator 602 through ultrasonic communication. In some embodiments, implantable device 604 can be configured to wirelessly communicate with one or more other implantable devices through ultrasonic communication. In some embodiments, implantable device 604 can be implanted within a subject such as a patient and interrogator 602 can be a separate device that is external to (i.e., non-implanted) or fully-implanted in the subject.

In some embodiments, to enable implantable device 604 to be powered and operated using ultrasonic waves, implantable device 604 can include the following device components: an ultrasonic transducer circuit 606, a modulation and demodulation circuit 612, a stimulation circuit 614, a detection circuit 616, a controller circuit 620, and a power circuit 630. In some embodiments, one or more of these device components can be implemented as a digital circuit, an analog circuit, or a mixed-signal integrated circuit depending on their operations. For example, controller circuit 620 may include a microprocessor, a finite state machine (FSM), a field programmable gate array (FPGA), or a microcontroller.

In some embodiments, ultrasonic transducer circuit 606 includes an ultrasonic transducer 608 coupled to a matching network 610. In some embodiments, ultrasonic transducer circuit 606 does not include matching network 610. In some embodiments, ultrasonic transducer 608 can be configured to receive ultrasonic waves from interrogator 602 and convert energy from the received ultrasonic waves into an electrical signal to power one or more device components of implantable device 604. In some embodiments, the electrical signal can be generated by ultrasonic transducer 608 because vibrations of ultrasonic transducer 608 caused by the received ultrasonic waves induce a voltage across the electric terminals of ultrasonic transducer 608, which causes an electrical current to flow.

In some embodiments, as described above, power from the received ultrasonic waves can be used by implantable device 604 to power its device components; accordingly, these ultrasonic waves are sometimes referred to herein as powering ultrasonic waves. In some embodiments, the received ultrasonic waves can encode information including operating mode commands for operating the implantable device; accordingly, these ultrasonic waves are sometimes referred to herein as communication ultrasonic waves. In some embodiments, similar to how powering ultrasonic waves can be processed, the communication ultrasonic waves can be received by ultrasonic transducer 608 to generate an electrical signal having an electrical current that flows through ultrasonic transducer 608. In some embodiments, the generated electrical signal encodes the operating mode commands in the electrical current. In some embodiments, the same ultrasonic waves can be configured to both power implantable device 604 and to encode information for transmitting to implantable device 604. In some embodiments, as described below with respect to FIG. 2, each operating mode command can include one or more ultrasound pulses and each ultrasound pulse may include one or more carrier cycles of ultrasonic waves.

In some embodiments, ultrasonic transducer circuit 606 includes a plurality of ultrasonic transducers coupled to a plurality of corresponding matching networks. By including at least two ultrasonic transducers, implantable device 604 can be configured to be powered by electrical signals generated by the at least two ultrasonic transducers to more efficiently and consistently extract power provided by interrogator 602, according to some embodiments. In some embodiments, implantable device 604 can be configured to harvest power from one or more ultrasonic transducers selected from the plurality of ultrasonic transducers. For example, implantable device 604 may select an ultrasonic transducer that provides the highest power or the most consistent power.

For example, a host of factors such as an orientation of ultrasonic transducer or intervening biological material between ultrasonic transducer 608 and an ultrasonic wave source interrogator 602 may significantly reduce the power receivable at ultrasonic transducer 608. By adding one or more additional ultrasonic transducers, reduced power receivable at a single ultrasonic transducer (e.g., ultrasonic transducer 608) may be less likely to negatively impact operations of implantable device 604.

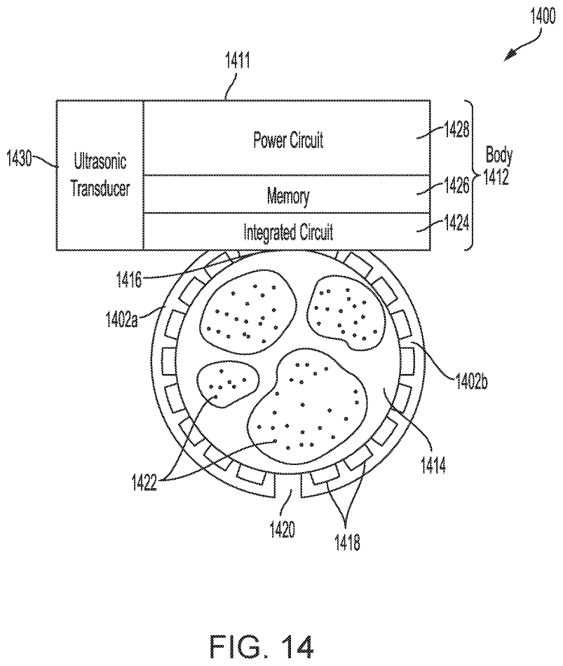

In some embodiments, including at least two ultrasonic transducers may enable implantable device 602 to be more reliably controlled using ultrasonic waves. For example, implantable device 602 may be configured to compare the signal strength of the at least two ultrasonic transducers and select the signal having a highest signal strength to operate implantable device 602. In some embodiments, implantable device 602 can use a selected ultrasonic transducer to receive communication from (i.e., during downlink) and to backscatter information on (i.e., during uplink). In some embodiments, implantable device 602 can select a first ultrasonic transducer from the at least two ultrasonic transducers to receive ultrasonic communications for downlink ultrasonic communication and select a second ultrasonic transducer from the at least two ultrasonic transducers to backscatter encode information for uplink ultrasonic communications. In some embodiments, implantable device 602 can be configured to perform beamforming with the at least two ultrasonic transducers to improve the signal to noise ratio of the uplink and downlink ultrasonic communications. In some embodiments, one or more of these ultrasonic transducers can be a micro machined ultrasonic transducer, such as a capacitive micro-machined ultrasonic transducer (CMUT) or a piezoelectric micro-machined ultrasonic transducer (PMUT), or can be a bulk piezoelectric transducer. Additionally implementations of ultrasonic transducer 608 are described below with respect to FIG. 14.

In some embodiments, matching network 610 can be an electronic circuit configured to select an impedance match between the electrical impedance of ultrasonic transducer 608 and the electrical impedance of implantable device 604 (e.g., power circuit 630) to reduce signal reflection. In some embodiments, matching network 610 can be implemented in various configurations of one or more circuit elements such inductors, capacitors, resistors, diodes, transistors, or any combination thereof. For example, matching network 610 may be implemented as a plurality of capacitors connected in parallel and coupled to a plurality of corresponding switches. By controlling which of the switches open or close, matching network 610 may control how the plurality of capacitors is charged to select the impedance. In some embodiments, matching network 610 can be configured to enable the electrical signal generated by ultrasonic transducer 608 to bypass the plurality of capacitors via a separate wire controlled by a switch.

In some embodiments, to enable implantable device 604 to be powered using ultrasonic waves, power circuit 630 can include a power recovery circuit 632 electrically coupled to a regulation circuit 638. In some embodiments, power recovery circuit 632 can be configured to receive and process the electrical signal generated by ultrasonic transducer circuit 606. In some embodiments, power recovery circuit 632 can include a rectifying circuit (e.g., an active rectifier) to convert the electrical signal in an AC form to a DC form where the converted electrical signal may be associated with a first voltage (i.e., the supply voltage of the received ultrasonic waves).

In some embodiments, due to health hazards in propagating high-powered waves through biological tissue of the subject, government regulations may limit the amount of power (e.g., 720 mW/cm²) provided by ultrasonic waves transmitted by interrogator 602. Therefore, the first voltage derived from the received ultrasonic waves may not be high enough to operate the electronic components of implantable device 104. For example, transistors used in complementary metal-oxide-semiconductor (CMOS) technology may require a minimum of about 2 Volts to operate the transistors.

In some embodiments, to provide a higher first voltage to operate the electronic components implantable device 602, the powering ultrasonic waves can be transmitted as a pulse width modulated (PWM) signal. In some embodiments, by transmitting the powering ultrasonic waves as the PWM signal, interrogator 602 can be configured to provide short, high intensity pulses such that the average intensity stays within the regulation limits and to provide higher instantaneous power to generate a higher first voltage. In some embodiments, the interrogator can be configured to control an instantaneous intensity and/or a pulse width (e.g., example ultrasonic wave settings) of the PWM signal to control the power provided by the powering ultrasonic waves.

In some embodiments, to enable implantable device 604 to be powered by these ultrasonic waves, power conveyor circuit 634 can include a charge pump configured to convert the first voltage to a second voltage greater than the first voltage. In some embodiments, the charge pump can include a plurality of coupled capacitors controlled by one or more switches to generate the second voltage. In some embodiments, the charge pump can achieve conversion gains of at least 1×, 2×, 3×, or 4×. In some embodiments, the magnitude of the second voltage can be controlled based on a switching frequency of the one or more switches.

As discussed above, power provided by the received ultrasonic waves can be inconsistent due to a host of factors including, for example, an implant depth of implantable device 604 or intervening biological material between ultrasonic transducer 608 and the ultrasonic wave source, e.g., interrogator 602. Accordingly, in some embodiments, to provide more consistent power to implantable device 604, power recovery circuit 632 can include an energy storage device 636 coupled to power conveyor circuit 634. In some embodiments, the energy storage device includes a battery or a storage capacitor. In some embodiments, to retain the small form factor of implantable device 604, the energy storage device can be configured as a storage capacitor.

In some embodiments, the storage capacitor can have a capacitance that is at least 0.1 μF, at least 0.25 μF, at least 0.5 μF, at least 1 μF, at least 2 μF, at least 4 μF, or at least 8. In some embodiments, the storage capacitor can have a capacitance that is less than 10 μF, less than 8 μF, less than 4 μF, less than 2 μF, less than 1 μF, less than 0.5 μF, or less than 0.25 μF. For example, the storage capacitor may have a capacitance in the range of 0.1-10 μF such as in the range of 0.5-2 μF. In some embodiments, the storage capacitor can have a capacitance that is about 1 μF.

In some embodiments, energy storage device 636 can be configured to operate in at least two power modes to enable implantable device 604 to more efficiently utilize power of received ultrasonic waves and to provide more consistent power. In some embodiments, the power modes include a charging mode in which a portion of power of the received ultrasonic waves can be conveyed to energy storage device 636 capable of storing the energy. In some embodiments, power conveyor circuit 634 can be configured to charge energy storage device 636 based on the generated first voltage. In some embodiments, the power modes include a discharging mode in which a portion of energy stored at energy storage device 636 is discharged to convey power from energy storage device 636 to provide additional power to other device components (e.g., stimulation circuit 614, detection circuit 616, or controller circuit 620, etc.) of implantable device 604. In some embodiments, the power flow to and from energy storage device 636 can be routed through power conveyor circuit 634.

In some embodiments, regulation circuit 638 can be configured to regulate the output voltage (e.g., the second voltage) generated by power conveyor circuit 634 to provide regulated voltages to one or more circuit loads of implantable device 604. In some embodiments, where power conveyor circuit 634 includes a charge pump, regulation circuit 638 can be configured to remove or reduce potential voltage ripples caused by operating switches of the charge pump. In some embodiments, regulation circuit 638 includes a DC voltage regulator (e.g., a low-dropout (LDO) regulator) to regulate a voltage supplied to digital circuit loads of implantable device 604. In some embodiments, regulation circuit 638 includes a DC voltage regulator (e.g., a low-dropout (LDO) regulator) to regulate a voltage supplied to digital circuit loads of implantable device 604. In some embodiments, regulation circuit 638 includes an AC voltage regulator (e.g., a low-dropout (LDO) regulator) to regulate a voltage supplied to analog circuit loads of implantable device 604.

In some embodiments, modulation and demodulation circuit 612 can include a demodulation circuit configured to demodulate the electrical signal generated by ultrasonic transducer circuit 606 to extract information encoded in the received ultrasonic waves. In some embodiments, the demodulation circuit can transmit the extracted information including an instruction to controller circuit 620 configured to control how implantable device 604 operates based on the instruction.

In some embodiments, to enable implantable device 604 to wireless communicate information with interrogator 602, modulation and demodulation circuit 612 can include a modulation circuit configured to encode the information using ultrasonic backscatter. This information is generated by implantable device 604 and, for ease of explanation, will sometimes be referred to as device information in the following descriptions.

In general, when implantable device 604 is embedded within a subject, the ultrasonic waves (including carrier waves) emitted by an ultrasonic transceiver of interrogator 602 will pass through biological tissue before being received by ultrasonic transducer circuit 606 of implantable device 604. As described above, the carrier waves cause mechanical vibrations on ultrasonic transducer 608 (e.g., a bulk piezoelectric transducer) to generate a voltage across ultrasonic transducer 608, which then imparts an electrical current to flow to the rest of implantable device 604. In some embodiments, the electrical current flowing through ultrasonic transducer 608 causes ultrasonic transducer circuit 606 to emit backscatter ultrasonic waves corresponding to the received ultrasonic waves.

In some embodiments, the modulation circuit 612 can be configured to modulate the electrical current flowing through ultrasonic transducer 608 to encode the device information, which causes the resulting ultrasonic backscatter waves to also encode the device information. Accordingly, the ultrasonic backscatter emitted from implantable device 604 can encode the device information related to implantable device 604. In some embodiments, the modulation circuit can include one or more switches, such as an on/off switch or a field-effect transistor (FET). An example FET that may be used with some embodiments of implantable device 604 includes a metal-oxide-semiconductor field-effect transistor (MOSFET). In some embodiments, the modulation circuit can be configured to alter the impedance of an electrical current flowing through ultrasonic transducer 608, and variation in the flowing electrical current flowing encodes the information.

As described above, ultrasonic power provided by interrogator 602 can only be increased by so much and needs to be below the thresholds deemed safe by regulatory bodies. However, due to misalignment between ultrasonic transducer 608 and the US beam emitted by interrogator 602, power supplied by interrogator 602 may be not effectively received and by ultrasonic transducer 608. In some embodiments, implantable device 604 can utilize ultrasonic communications by embedding implant signals or information within the ultrasonic backscatter to enable interrogator 602 to better track implantable device 604. For example, as described above with respect to FIG. 5, the ultrasonic backscatter can be received by interrogator 602 and deciphered to extract the device information encoded in the ultrasonic backscatter. Then, interrogator 602 can compare the extracted information with a predetermined pattern associated with implantable device 604 and/or determine a signal strength from the extracted information to change a beam focus of the emitted US beam to increase alignment with ultrasonic transducer 608 of implantable device 604, according to some embodiments. In some embodiments, the ultrasonic backscatter can be received by an interrogator that may be the same or different than interrogator 602 that transmitted the ultrasonic waves received by ultrasonic transducer 608.

In some embodiments, detection circuit 616 can be configured to interface with one or more sensors 640A-C to measure or detect one or more physiological conditions of the subject. In some embodiments, detection circuit 616 can include a driver configured to provide current to the one or more sensors 640A-C and receive generated signals from the one or more sensors 640A-C. In some embodiments, a received signal can include information representative of a detected physiological condition or representative of a measured physiological condition. In some embodiments, detection circuit 616 can be configured to transmit the information to controller circuit 620.

In some embodiments, one or more of sensors 640A-C can be located inside implantable device 604 or coupled to the exterior of implantable device 604. In some embodiments, implantable device 604 includes at least two sensors 640A-C. In some embodiments, the one or more physiological conditions can include temperature, pH, pressure, heart rate, strain, oxygen tension, a presence of an analyte, or an amount of the analyte. For example, the analyte may be oxygen or glucose.

In some embodiments, sensors 640A-C can include an optical sensor. In some embodiments, the optical sensor comprises a light source and an optical detector. In some embodiments, the optical sensor detects blood pressure or a pulse. In some embodiments, the optical sensor comprises a matrix comprising a fluorophore or luminescent probe, and wherein fluorescence intensity or fluorescence lifetime of the fluorophore depends on the amount of the analyte. In some embodiments, the optical sensor is configured to perform near-infrared spectroscopy. In some embodiments, the optical sensor detects glucose.

In some embodiments, sensors 640A-C can include a potentiometric chemical sensor or an amperometric chemical sensor. In some embodiments, the sensor detects oxygen, pH, or glucose. In some embodiments, sensors 640A-C can include a temperature sensor. In some embodiments, the temperature sensor is a thermistor, a thermocouple, or a proportional to absolute temperature (PTAT) circuit. In some embodiments, sensors 640A-C can include a pressure sensor. In some embodiments, the pressure sensor is a microelectromechanical system (MEMS) sensor. In some embodiments, detection circuit 616 is configured to measure blood pressure or a pulse. In some embodiments, sensors 640A-C can include a strain sensor.

In some embodiments, detection circuit 616 can be configured to interface with, for example, sensor 640C to detect an electrophysiological signal from a nerve or a targeted subset of nerve fibers within the nerve, as will be further explained below with respect to FIG. 14. In some embodiments, sensor 6140C can include electrode pads, which may be the same or different from electrode pads 642 operated by stimulation circuit 614. In some embodiments, detection circuit 616 can be configured to record neural activity of a nerve or the targeted subset of nerve fibers based on the detected electrophysiological signal.

In some embodiments, one or more techniques such as computational modeling (e.g., finite element models), inverse source estimation, multipole (e.g., tripole) neural recording, velocity-selective recording, or beamforming can be implemented by detection circuit 116 (alone or in conjunction with controller circuit 120) to selectively target the subset of nerve fibers. See, for example, Taylor et al., Multiple-electrode nerve cuffs for low-velocity and velocity selective neural recording, Medical & Biological Engineering & Computing, vol. 42, pp. 634-643 (2004); and Wodlinger et al., Localization and Recovery of Peripheral Neural Sources with Beamforming Algorithms, IEEE Transactions on Neural Systems and Rehabilitation Engineering, vol. 17, no. 5, pp. 461-468 (2009).

In some embodiments, detection circuit 616 can be configured to operate the plurality of electrodes of sensor 640C for targeted detection of the electrophysiological signal. For example, sensor 640C may be a curved member that extends from implantable device 604, as further described below with respect to FIG. 14. In some embodiments, detection circuit 616 can analyze the electrophysiological signal detected by all or a subset of the electrode pads to determine the subset of nerve fibers within the nerve that are transmitting the electrophysiological signal. Certain nerves may transmit compound electrophysiological signal (or compound action potentials), which is the sum of the electrophysiological signals (or action potentials) simultaneously transmitted by two or more different subsets of nerve fibers. Based on the electrophysiological signal detected by the plurality of electrode pads, detection circuit 616 may be able to determine which subset of nerve fibers transmits which electrophysiological signal. In some embodiments, data received from interrogator 602 (such as temperature data, or data related to an analyte concentration or other physiological condition) is further used to determine which subset of nerve fibers transmits the electrophysiological signal.

For example, in some embodiments, detection circuit 616 may be configured to selectively detect an electrophysiological signal from a targeted subset of nerve fibers using velocity-selective recording, which may be combined with multipolar (e.g., tripolar) recording (which can include any number of tripoles within the plurality of electrodes on one or more curved members).

Beamforming can additionally or alternatively be used to detect the electrophysiological signals from the targeted subset of nerve fibers. A portion of or all of the electrode pads of one or more curved members can detect the electrophysiological signal from the nerve, and detection circuit 616 can determine the cross-sectional location of the transmitted signal within the nerve based on the differences in electrophysiological signal detected by a portion or all of the electrode pads of the one or more curved members.

In some embodiments, stimulation of one or more nerves at a location separate from the location of implantable device 604 can result in a modulation of the electrophysiological signal at the location of implantable device 604. The modulation of the electrophysiological signal detected at different subsets of nerve fibers within the nerve in electrical communication with the electrode pads (e.g., electrode pads 642) of implantable device 604 can be the result of stimulation in different distant nerves. For example, stimulation of the splenic nerve can result in modulation of an electrophysiological signal detected from first subset of nerve fibers within the vagus nerve, and stimulation of a renal nerve can result in modulation of an electrophysiological signal detected from a second subset of nerve fibers within the vagus nerve. Therefore, an implantable device positioned on the vagus nerve can detect an electrophysiological signal from the first subset of nerve fibers to monitor stimulation of the splenic nerve, and a second subset of nerve fibers to monitor stimulation of the renal nerve.

In some embodiments, stimulation circuit 614 can be configured to emit a targeted electrical pulse to a subset of nerve fibers within the nerve by selectively activating one or more electrode pads 642 connected to the subset of nerve fibers. In some embodiments, implantable device 604 can include one or more curved members that electrically connect stimulation circuit 614 to electrode pads 642, as will be further described below with respect to FIG. 14.

In some embodiments, stimulation circuit 614 can be controlled by controller circuit 620 to operate electrode pads 642 or to selectively activate electrode pads 642. Selective activation can include, for example, activating a portion of electrode pads within the plurality of electrode pads 642 of one or more curved members and/or differentially activating all or a portion of the electrode pads within the plurality of electrode pads 642 of the one or more curved members. The plurality of electrodes can therefore be operated to steer the electrical pulse emitted by the plurality of electrode pads 642 to the target subset of nerve fibers. Techniques such as electrical field interference or multipolar stimulation (e.g., tripolar stimulation) can be used to target the electrical pulse to the subset of nerve fibers within the nerve, according to some embodiments. See, for example, Grossman, et al., Noninvasive Deep Brain Stimulation via Temporally Interfering Electrical Fields, Cell, vol. 169, pp. 1029-1041 (2017). Electrode pads 142 within one or more curved members can be selectively activated by controller circuit 120 to target the emitted electrical pulse to the subset of nerve fibers.

The subset of nerve fibers targeted by the emitted electrical pulse can be the same or different as the subset of nerve fibers from which the electrophysiological signal is detected by detection circuit 616. The one or more curved member configured to emit the targeted electrical pulse can be the same or different as the one or more curved members on implantable device 604 configured to detect the electrophysiological signal. The emitted targeted electrical pulse can stimulate the nerve at the position of implantable device 604. The subset of nerve fibers targeted by the electrical pulse can be the same or a different subset of nerve fibers for which the electrophysiological signal is selectively detected.

The subset of nerve fibers targeted by the electrical pulse emitted by implantable device 604 can be, for example, one or more (e.g., 2, 3, 4, or more) fascicles, or a portion of one or more (e.g., 2, 3, 4, or more) fascicles within the nerve. In some embodiments, the subset of nerve fibers comprises or consists of afferent nerve fibers within the nerve, or a subset of afferent nerve fibers within the nerve. In some embodiments, the subset of nerve fibers comprises or consists of efferent nerve fibers within the nerve, or a subset of efferent nerve fibers within the nerve. In some embodiments, the subset of nerve fibers comprises or consists of efferent nerve fibers within two or more fascicles within the nerve or afferent nerve fibers within two or more fascicles within the nerve.

Targeted stimulation of a subset of nerve fibers by emitting a targeted electrical pulse to the subset of nerve fibers can result in stimulation of a nerve at a location distant from the position of the nerve. The distant nerve stimulated by implantable device 604 depends on the subset of nerves at the position of implantable device 604 targeted by the electrical pulse emitted by the device. In some embodiments, implantable device 604 is positioned at a first nerve locus and is configured to stimulate a second nerve locus by emitting a targeted electrical pulse to a subset of nerve fibers within the first nerve locus that is associated with the second nerve locus. In some embodiments, the first nerve locus and the second nerve locus are separated by one or more nerve branch points or one or more synapses. In some embodiments, the second nerve locus is proximal to the brain relative to the first nerve locus, and in some embodiment the second nerve locus is distal from the brain relative to the first nerve locus. In some embodiments, the targeted subset of nerve fibers comprises or consists of afferent nerve fibers. In some embodiments, the targeted subset of nerve fibers comprises or consists of efferent nerve fibers.

In some embodiments, controller circuit 620 includes a command processor 622, a mode detector 626, and a memory 650. In some embodiments, memory 650 includes a non-transitory storage memory such as register memory, a processor cache, or Random Access Memory (RAM). In some embodiments, controller circuit 620 can be a digital circuit, an analog circuit, or a mixed-signal integrated circuit. Examples of controller circuit 120 may include a microprocessor, a finite state machine (FSM), a field programmable gate array (FPGA), and a microcontroller.

In some embodiments, mode detector 626 can be configured to determine an operating mode command from the ultrasonic waves received by ultrasonic transducer 608. In some embodiments, mode detector 626 can determine the operating mode command upon determining a correspondence to a pattern from a plurality of predetermined patterns 656 stored in memory 650. For example, the pattern may be a sequence of one or more pulses having specific ultrasonic wave properties such as an ultrasound pulse duration. In this example, mode detector 626 can match a portion of the operating mode command to one or more of predetermined patterns 656 to determine a matching pattern. In another example, the pattern may correspond to an ultrasound property such as a pulse duration, an amplitude, or a phase or frequency change. In this example, mode detector 626 may analyze the ultrasound property (e.g., the pulse duration) of the portion to determine a correspondence to a pattern. In some embodiments, the portion of the operating mode command can be a single pulse that indicates the start of the operating mode command. In other embodiments, the portion can be a sequence of ultrasound pulses.

In some embodiments, mode detector 626 can receive the ultrasonic waves as an electrical signal that has been generated (e.g., demodulated) by modulation and demodulation circuit 612 based on the ultrasonic waves received in ultrasonic transducer circuit 606. In some embodiments, mode detector 626 can include one or more detection circuits configured to detect one or more ultrasonic wave properties from the electrical signal. In some embodiments, one of these detection circuits can include a zero-crossing circuit configured to determine a pulse duration of each ultrasound pulse in the operating mode command. For example, the zero-crossing circuit can be configured to count and store a number of instances that a first portion of the electrical signal crosses a predefined voltage level within a predetermined number of clock cycles to determine a pulse duration. In some embodiments, the predefined voltage level is a voltage close to 0 V (e.g., less than 10 mV, less than 50 mV, less than 100 mV, or less than 200 mV).

In some embodiments, command processor 622 can be configured to set an operating mode of implantable device 604 to one operating mode from a plurality of predetermined operating modes 652 based on the operating mode command determined by mode detector 626. In some embodiments, command processor 622 can store the received operating mode command and associated instructions in memory 650 such as an instruction register. In some embodiments, command processor 622 can be configured to control implantable device 604 to enter an operating state corresponding to the operating mode based on the stored operating mode command. For example, command processor 622 may be implanted as a FSM or a program in a microcontroller that controls the operating states of implantable device 604 based on a current operating state and one or more detected inputs such as one or more received operating mode commands, one or more sensor values, or a combination thereof.

In some embodiments, command processor 622 can be configured to extract information from a portion of the operating mode command to configure various parameters or to select an operating mode. Information encoded in the ultrasonic waves emitted by the interrogator and received by the closed-loop implantable device can include, for example, instructions for starting or stopping neuromodulation, one or more calibration instructions, one or more updates to the operation software, and/or or one or more templates (such as template electrophysiological signals, one or more template electrophysiological signals, and/or one or more template stimulation signals). In some embodiments, command processor 622 can be configured to process and store the received instructions in memory 650. In some embodiments, command processor 622 can enter an operating mode from a plurality of operating modes based on one or more received operating mode commands. In some embodiments, the plurality of operating modes can include, for example, a mode to stimulate a nerve, a mode to record neural activity, or a mode to determine one or more physiological conditions. For example, if the operating mode command indicates that implantable device 604 should enter the neural stimulation mode, controller circuit 620 may be configured to control stimulation circuit 614 to stimulate specific nerve fibers or portions of the nerve.

In some embodiments, when command processor 622 controls implantable device 104 to enter the neural activity recording mode or a mode to determine one or more physiological conditions, command processor 622 may control detection circuit 616 to retrieve the device information (e.g., neural record or detected/measured physiological condition). In some embodiments, command processor 622 can be configured to retrieve command 654 associated with a current operating mode 652 to control operations of implantable device 604. For example, in the neural activity recording mode, command processor 622 may receive command 654 corresponding to the neural activity recording mode and issue command 654 to control detection circuit 616 to sample a neural activity (e.g., an example of device information) of a nerve. In some embodiments, upon retrieving the device information, command processor 622 can be configured to control modulation and demodulation circuit 612 based on command 654 to encode the device information in an ultrasonic backscatter, as described above.

FIG. 7 illustrates a method 700 for discovery an implantable device using ultrasonic waves, according to some embodiments. In some embodiments, the implantable device may be an example of implantable device 120 as described above with respect to FIG. 1. In some embodiments, one or more steps of method 700 can be performed by an interrogator such as interrogator 106 or 502 as described above with respect to FIGS. 1 and 5, respectively. For example, one or more steps of method 700 may be performed by implant tracker 517. For ease of explanation, various steps below of method 700 may refer to components of interrogator 502. In some embodiments, method 700 can be performed by a system including the interrogator in communication with one or more computing devices. For example, some of the computing intensive steps may be offloaded from the interrogator to the one or more computing devices to increase computation speed and efficiency.

In step 702, the interrogator emits an ultrasonic (US) beam to successively focus on a plurality of focal points. For example, an implant tracker (e.g., implant tracker 517) of the interrogator may control how the US beam is emitted through a command generator (e.g., command generator 514). In some embodiments, the interrogator includes a transducer array including a plurality of transducers that can be controlled by the interrogator through electronic beam forming to focus the US beam at a specific focal point. For example, the command generator may generate instructions to control the transducer array, as described above with respect to FIG. 5. In some embodiments, the plurality of focal points represent a steerable range of the US beam. In some embodiments, the steerable range may include a linear range. In other embodiments in which the transducer array can include a 2D array of transducers, the steerable range may include a 2D region.

In step 704, at each focal point of the plurality of focal points, the interrogator determines how likely the implantable device is located at the focal point. In some embodiments, the interrogator can perform steps 704A-C at each focal point of the plurality of focal points.

In step 704A, the interrogator holds the focused US beam at the focal point for a duration that permits an implantable device, if located at the focal point, to convert energy from ultrasonic waves of the US beam into electrical energy to enter a powered-on state from a powered-off state. In some embodiments, the duration can be a predefined period of time that is previously determined based on various factors including one or more of a strength of the US beam, a power requirement of the implantable device, an energy storage capacity of the implantable device, or an average or estimated maximum distance between the interrogator and the implantable device.

In step 704B, the interrogator receives backscattered ultrasonic waves corresponding to the US beam focused on the focal point. In some embodiments, the interrogator can operate a switch to toggle between transmitting the US beam and receiving ultrasonic backscatter. In some embodiments, the implantable device receiving the ultrasonic waves of the US beam can be configured to encode information in an ultrasonic backscatter emitted by the implantable device. For example, the implantable device may modulate an electric signal by digitally controlling a switch to shunt the ultrasonic transducer to encode the information. In some embodiments, the information may include a predetermined pattern that identifies the implantable device. In some embodiments, the predetermined pattern may be a square wave oscillation, by which the implantable device periodically shorts the piezo terminals of its one or more transducers for a predetermined period of time. In some embodiments, the predetermined pattern may be a sequence of digital data decoded by the interrogator, as described above with respect to digital data processing 312 of FIG. 3.

In step 704C, the interrogator compares the received backscattered ultrasonic waves with a predetermined pattern associated with the implantable device to be discovered to generate a score indicating how likely the backscattered ultrasonic waves comprise the predetermined pattern. For example, the implant tracker may store the predetermined pattern in a memory and compare the predetermined pattern with the backscattered ultrasonic waves. In some embodiments, the implant tracker may store a sequence of digital data corresponding to the predetermined pattern and decode the backscattered ultrasonic waves to determine whether the predetermined pattern is present in the backscattered ultrasonic waves. In some embodiments, the score can indicate whether or not the predetermined pattern of the implantable device is detected from the ultrasonic backscatter. In some embodiments, the interrogator can communicate (e.g., through a wired connection or a wireless connection) with one or more computing devices to generate the score.

In step 706, the interrogator determines a location of the implantable device from the plurality of focal points based on a plurality of scores generated for the plurality of corresponding focal points. In some embodiments, the implant tracker of the interrogator can estimate the location of the implantable device based on which focal points of the plurality of focal points have scores that are at least a predefined threshold or confidence level. For example, the interrogator may determine the location by computing one or more measures of central tendency such as the median, mode, or average of the focal points whose scores are at or above the predefined threshold (e.g., 80%, 90%, 95%, etc.). In some embodiments, the implant tracker can be configured to calculate a spectral centroid (i.e., a center of mass) of the scores across the plurality of focal points. In other words, the implant tracker may compute a weighted average of the scores across the plurality of focal points to identify an “average” focal point value representing the “center of mass” of the plurality of focal points with respect to the plurality of corresponding scores. In some embodiments, the interrogator can select a focal point from the plurality of focal point as representing the location of the implantable device.

In some embodiments, once the interrogator determines an estimated location of the implantable device, the interrogator can be configured to direct the US beam to a focal point closest to the estimated location to confirm that the implantable device is located at that focal point. For example, the interrogator can focus the US beam on the focal point selected from the plurality of focal points in determining the estimated location in step 706. In some embodiments, the interrogator can analyze an ultrasonic backscatter received while the US beam is focused on the selected focal point to confirm that the implantable device is located at the selected focal point. For example, the interrogator may compare a signal strength extracted from the ultrasonic backscatter with a predetermined threshold value. In some embodiments, the interrogator can maintain the US beam at the selected focal point in response to confirming that the implantable device is located at the selected focal point. Otherwise, the interrogator can steer the US beam to refocus on one or more focal points from a second plurality of focal points in response to confirming that the implantable device is not located at the selected focal point, according to some embodiments. For example, the one or more focal points may be selected from the plurality of focal points of step 702.

In some embodiments, once the interrogator discovers the implantable device and determines the location of the implantable device, the interrogator can enter a tracking mode in which the interrogator determines and maintains alignment between the US beam and the implantable device, as will be further described below with respect to FIGS. 8-11.

FIG. 8 illustrates a diagram 800 showing example operating logic of an interrogator (e.g., interrogator 106 of FIG. 1 or interrogator 502 of FIG. 5) to effectively track and power an implantable device using ultrasonic waves, according to some embodiments. As discussed above, a controller circuit (e.g., controller circuit 512) of the interrogator can be configured to implement a finite state machine (FSM) to control operations of the interrogator. For example, an implant tracker (e.g., implant tracker 517) of the interrogator may implement the FSM. For example, diagram 800 shows a Moore state machine. As shown in diagram 800, the FSM may include a plurality of operating states 802-806 for tracking the implantable device. While the FSM is shown as being a Moore machine, the interrogator can be configured to control its operating logic according to other types of FSMs. For example, instead of the Moore machine, the FSM may be implemented as a Mealy state machine, a Harel state machine, or a Unified Modeling Language (UML) state machine.

In operating state 802, the interrogator can be configured to establish a synchronization state with the implantable device. In some embodiments, the interrogator steers its US beam to focus on a plurality of focal points to determine a focal point at which a signal strength determined from received ultrasonic backscatter is above a predetermined synchronization threshold. As shown, if the determined signal strength is below the predetermined threshold, the interrogator remains in operating state 802. Once the signal strength meets or exceeds the predetermined threshold, the interrogator enters operating state 804.

In operating state 804, the interrogator can be configured to track a location of the implantable device. In some embodiments, the interrogator adjusts where the US beam is being focused to maximize the signal strength of a signal extracted from received ultrasonic backscatter. In some embodiments, the interrogator can be configured to stay in operating state 804 and adjust the position of the focal point until a corresponding signal strength is no longer increasing, i.e., a local maximum has been found. Once the signal strength has been maximized, the interrogator enters operating state 806.

In operating state 806, the interrogator maintains the US beam to focus on the focal point that resulted in the maximum signal strength in operating state 804. In some embodiments, this maximum signal strength can represent a steady-state threshold. To provide consistent power and reliable ultrasonic communications between the interrogator and the implantable device, the interrogator is configured to monitor the signal strength of the signal received in ultrasonic backscatter. If the monitored signal strength is determined to be within a predetermined range of the steady-state threshold, then the interrogator maintains the US beam focus. Otherwise, if the monitored signal strength falls outside of the range of the steady-state threshold, the interrogator reenters operating state 804 to track the location of the implantable device.

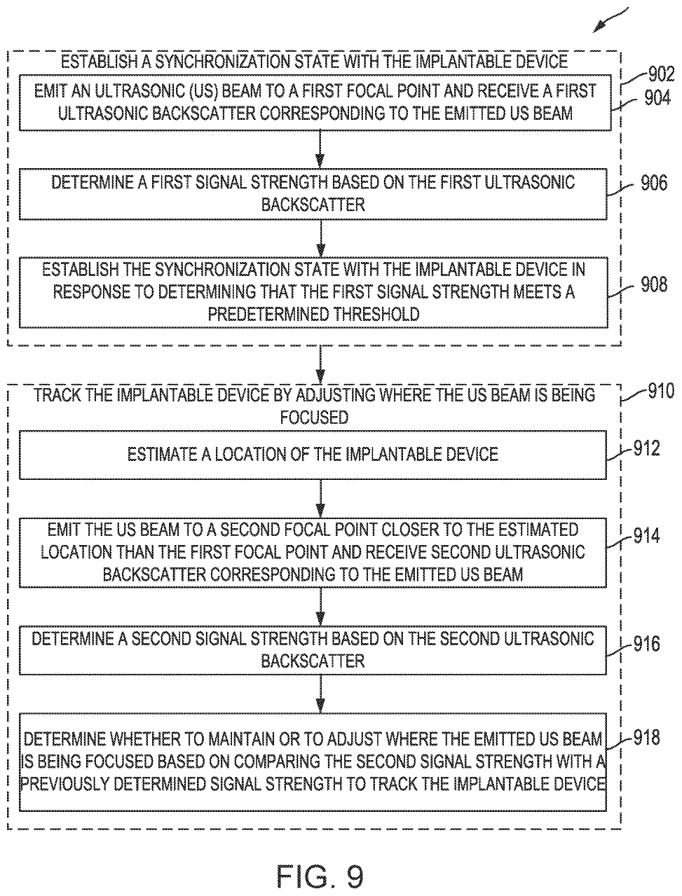

FIG. 9 illustrates a method 900 for tracking an implantable device powered using ultrasonic waves to maintain power supplied to the implantable device, according to some embodiments. In some embodiments, the implantable device may be an example of implantable device 120 as described above with respect to FIG. 1. In some embodiments, one or more steps of method 900 can be performed by an interrogator such as interrogator 106 and 502 as described above with respect to FIGS. 1 and 5, respectively. For example, one or more steps of method 900 can be performed by implant tracker 517 of implantable device 502, as described above with respect to FIG. 5. In some embodiments, method 900 can be performed by a tracking system including the interrogator in communication with one or more computing devices. For example, some of the computing intensive steps may be offloaded from the interrogator to the one or more computing devices to increase computation speed and efficiency. For ease of explanation, various steps below of method 900 may refer to components of interrogator 502.

In step 902, the interrogator establishes a synchronization state with the implantable device. In some embodiments, step 902 includes steps 904-908.

In step 904, the interrogator emits an ultrasonic (US) beam to a first focal point and receive a first ultrasonic backscatter corresponding to the emitted US beam. As described above, when the ultrasonic waves of the US beam contacts the implantable device, the ultrasonic waves are scattered and part of its energy is radiated in all spatial directions including back towards the interrogator. In some embodiments, the implantable device can be configured to modulate an electrical signal to encode information within the ultrasonic backscatter.

In step 906, the interrogator determines a first signal strength based on the first ultrasonic backscatter. In some embodiments, the implant tracker of the interrogator can be configured to extract an implant signal from the ultrasonic backscatter and determine its signal strength. As described above with respect to FIG. 3, the implant signal may correspond to signal modulation performed by the implantable device to encode implant data.

In some embodiments, the implant tracker can cancel signal interference or environmental noise from the received backscattered ultrasonic waves to extract the implant signal. In some embodiments, the implant tracker can perform interference cancellation by compare a first portion of the ultrasonic backscatter that includes the implant signal with a second portion of the ultrasonic backscatter that does not include the implant signal to extract the implant signal. For example, the implant signal may subtract the second portion (corresponding to passive backscatter with no implant modulation) from the first portion (corresponding to active backscatter with implant modulation) to cancel out environmental noise or interference.

In some embodiments, the implant tracker can be configured to determine the signal strength from the implant signal extracted from the ultrasonic backscatter. In some embodiments, the implant tracker can determine the signal strength by determining a modulation depth or an amplitude variation of the extracted signal. For example, the implant tracker may determine the amplitude variation as a percentage of amplitude variation.

In step 908, the interrogator establishes the synchronization state with the implantable device in response to determining that the first signal strength meets a predetermined threshold. For example, the predetermined threshold may be a minimum amplitude threshold.

In step 910, once the synchronization state is established, the interrogator tracks the implantable device by adjusting where the US beam is being focused. In other words, the interrogator tracks a location of the implantable device such that a focal point of the US beam is in alignment with the location of the implantable device. In some embodiments, tracking the implantable device is critical to maintain sufficient power provided by the US beam to the implantable device and to achieve reliable bi-directional ultrasonic communications between the interrogator and the implantable device. By tracking the implantable device, the interrogator can be configured to operate according to regulatory guidelines of maximum allowable power directed at in-body devices. In some embodiments, step 910 includes steps 912-918.

In step 912, the interrogator estimates a location of the implantable device. In some embodiments, the interrogator can be configured to estimate the location based on the first ultrasonic backscatter. In some embodiments, the interrogator determines a direction in which to adjust a position of the first focal point based on receive beamforming. In some embodiments, the interrogator can determine the estimated location based on one or more predetermined portions of the first ultrasonic backscatter. In some embodiments, the interrogator can determine the estimated location based on one or more ultrasonic backscatters received after the first ultrasonic backscatter.

In step 914, the interrogator emits the US beam to a second focal point closer to the estimated location than the first focal point and receives second ultrasonic backscatter corresponding to the emitted US beam.

In step 916, the interrogator determines a second signal strength based on the second ultrasonic backscatter received in step 914. For example, similar to how the first signal strength may be determined from the first ultrasonic backscatter in step 906, the implant tracker of the interrogator may extract a second implant signal from the second ultrasonic backscatter and determine the second signal strength from the second extracted implant signal.

In step 918, the interrogator determines whether to maintain or to adjust where the emitted US beam is being focused based on comparing the second signal strength with a previously determined signal strength to track the implantable device. In some embodiments, the interrogator can compare the second signal strength with the previously determined first signal strength to determine whether to maintain or adjust the focus of the US beam. For example, if the second signal strength is greater than the first signal strength, the interrogator can adjust the focal point in a direction of the second focal point. In another example, if the second signal strength is less than the previously determined signal strength, than the interrogator can maintain the focus at the first focus point to maintain an acceptable level of synchronization or alignment between the US beam and the implantable device.

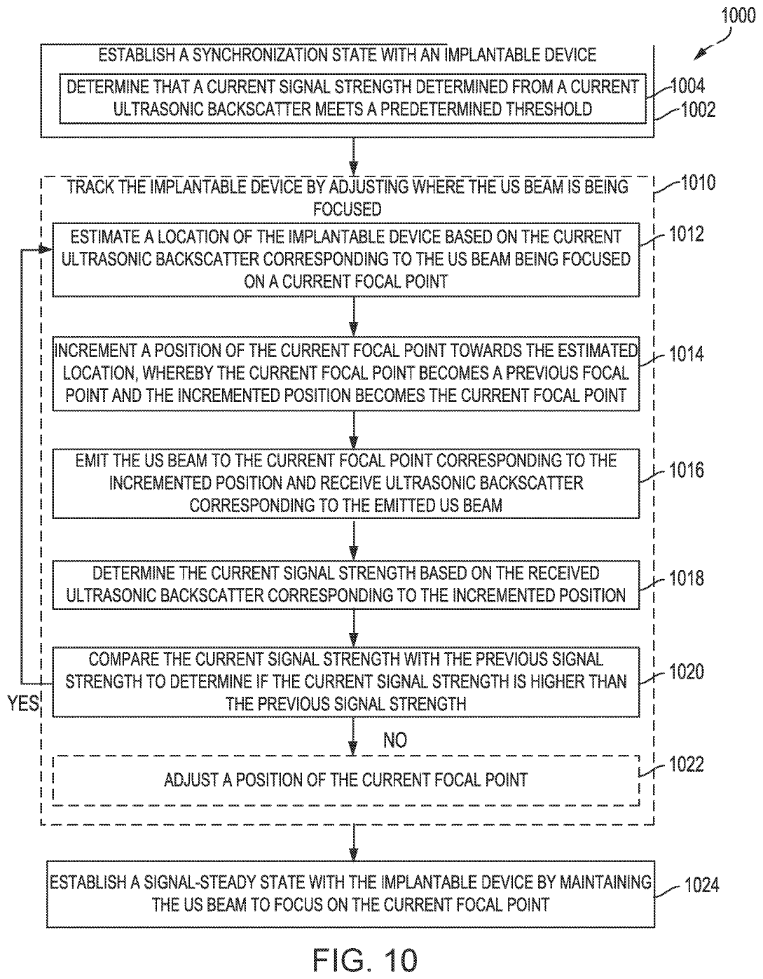

FIG. 10 illustrates a method 1000 for tracking an implantable device powered using ultrasonic waves to effectively maintain power supplied to the implantable device, according to some embodiments. In some embodiments, method 1000 illustrates additional details that expands upon step 910, as described above with respect to FIG. 9. In some embodiments, one or more steps of method 1000 can be performed by an implant tracker (e.g., implant tracker 517) of interrogator 502, as described above with respect to FIG. 5.

In step 1002, the interrogator established a synchronization state with an implantable device, as described above with respect to step 902 of FIG. 9. In particular, step 1002 includes step 1004, at which the interrogator determines that a current signal strength determined from a current ultrasonic backscatter meets a predetermined threshold. As described above with respect to FIG. 9, an implant tracker of the interrogator may be configured to determine a signal strength from a received ultrasonic backscatter by extracting an implant signal from the ultrasonic backscatter and determining the signal strength of the extracted implant signal.

In step 1010, the interrogator tracks the implantable device by adjusting where the US beam is being focused. In some embodiments, step 1010 includes steps 1012-1020.

In step 1012, the interrogator estimates a location of the implantable device based on the current ultrasonic backscatter corresponding to the US beam being focused on a current focal point. For example, the implant tracker of the interrogator can estimate the location using receive beamforming. In some embodiments, the estimated location can be represented by an estimate angle to adjust where the US beam is being focused. In some embodiments, the estimated location can be represented by an estimate angle of the US beam with respect to the transducer array of the interrogator. In some embodiments, the implant tracker can determine an estimate angle that represents an estimate of the location based on using receive beamforming. For example, by directing the US beam and its respective focal point in the direction indicated by the estimate angle, the distance between the true location of the implantable device and the focal point of the US beam can be reduced.

In step 1014, the interrogator increments a position of the current focal point towards the estimated location, whereby the current focal point becomes a previous focal point and the incremented position becomes the current focal point. In some embodiments, the position can be incremented by a predetermined amount. For example, this amount may be at least 0.1 mm, 0.2 mm, 0.25 mm, 0.5 mm, 0.6 mm. For example, this amount may be less than 0.7 mm, 0.6 mm, 0.5 mm, 0.4 mm, 0.25 mm, or 0.2 mm. In some embodiments where the estimated location is represented by an estimated angle, the interrogator can be configured to increment the position of the current focal point in the direction indicated by the estimated angle. Accordingly, by estimating the location of the implantable device and controlling where the US beam is being focused, the interrogator can reduce the number of focal points that need to be searched and increases searching speed and efficiency.

In step 1016, the interrogator emits the US beam to the current focal point corresponding to the incremented position and receives ultrasonic backscatter corresponding to the emitted US beam.

In step 1018, the interrogator determines the current signal strength based on the received ultrasonic backscatter corresponding to the incremented position. In some embodiments, as described above with respect to step 906 of FIG. 9, the interrogator can extract an implant signal (i.e., a signal embedded by the implantable device in the ultrasonic backscatter) from the backscatter and determine the signal strength of the extracted implant signal.

In step 1020, the interrogator compares the current signal strength with the previous signal strength to determine if the current signal strength is higher than the previous signal strength. In other words, the interrogator may determine whether incrementing the position of beam focus from the previous focal point to the current focal point increased the signal strength and hence improves the alignment between the interrogator and the implantable device.

In some embodiments, if the current signal strength increases, then method 1000 returns to step 1012 in which the interrogator continues to adjust the position of the focal point. In some embodiments, once the current signal strength is determined to no longer increase or to decrease, the interrogator determines that a local maximum signal strength has been determined and that the associated focal point is closest to the location of the implantable device. In some embodiments, the interrogator optionally performs step 1022, at which a position of the current focal point is adjusted. For example, the interrogator may revert the incremented position of the current focal point by half the increment to account for the discrete incremented amount.

In step 1024, the interrogator established a signal-steady state with the implantable device by maintain the US beam to focus on the current focal point.

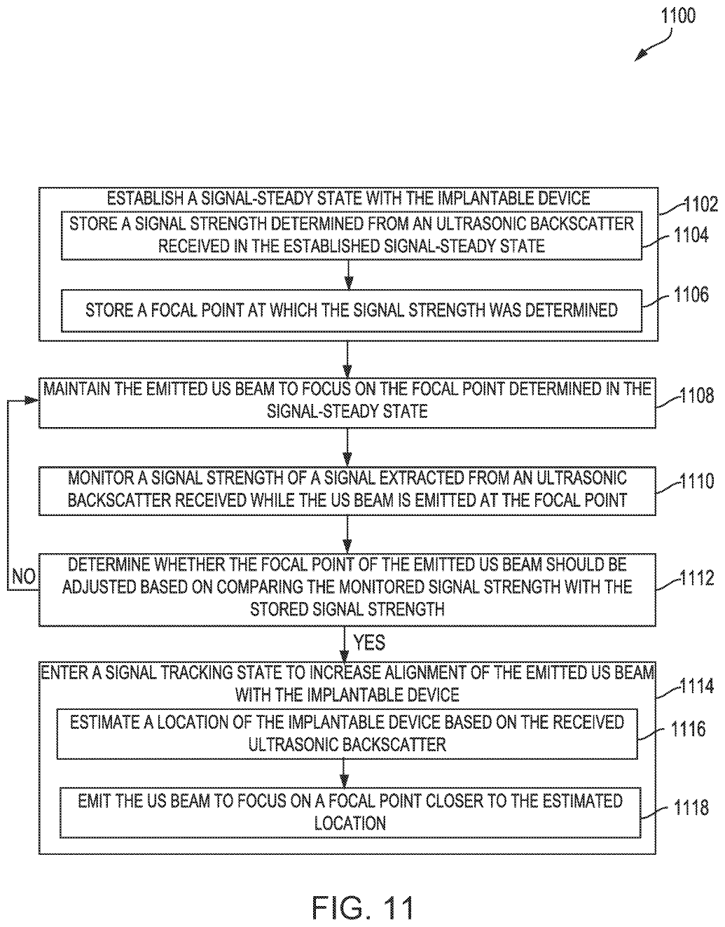

FIG. 11 illustrates a method 1100 for tracking an implantable device powered using ultrasonic waves to effectively maintain power supplied to the implantable device, according to some embodiments. In some embodiments, method 1100 illustrates additional details that expands upon step 1024, as described above with respect to FIG. 10. In some embodiments, one or more steps of method 1100 can be executed by an implant tracker (e.g., implant tracker 517) of interrogator 502, as described above with respect to FIG. 5.

In step 1102, the interrogator establishes a signal-steady state with the implantable device. In some embodiments, step 1102 includes steps 1104-1106.

In step 1104, the interrogator stores a signal strength determined from an ultrasonic backscatter received in the established signal-steady state. In other words, the interrogator can be configured to store the maximum signal strength that was determined while tracking the implantable device as described above with respect to FIG. 10.

In step 1106, the interrogator stores a focal point at which the signal strength was determined in step 1104. In some embodiments, the focal point corresponds to where the US beam emitted by the interrogator was targeted.

In step 1108, the interrogator maintains the emitted US beam to focus on the focal point determined in the signal-steady state.

In step 1110, the interrogator monitors a signal strength of a signal extracted from an ultrasonic backscatter received while the US beam is emitted at the focal point. For example, similar to step 906 of FIG. 9, an implant tracker of the interrogator may be configured determine the signal strength based on extracting an implant signal from the ultrasonic backscatter.

In step 1112, the interrogator determines whether the focal point of the emitted US beam should be adjusted based on comparing the monitored signal strength with the stored signal strength. In some embodiments, if the interrogator determines that the monitored signal strength does not fall below a predetermined threshold of the stored signal strength, method 1100 returns to step 1108, at which the focal point of the emitted US beam is maintained. Otherwise, method 1100 proceeds to step 1114. In some embodiments, the interrogator can determine whether the focal point should be adjusted based on whether the monitored signal strength decreases below a percentage of the stored signal strength. As described above, the stored signal strength represents a previously identified local maximum. Accordingly, the interrogator can adjust the alignment between the interrogator and the implantable device to counteract the movement of the subject, which causes the location of the implantable device to change.

In some embodiments, in addition to monitoring the signal strength to counteract the movement of the implantable device, the interrogator can be configured to monitor a movement of the interrogator to determine whether and how to adjust the focal point of the emitted US beam to counteract movement of the interrogator. For example, the interrogator may include one or more of an inertial movement unit (IMU), an accelerometer, or a gyroscope to detect and measure a movement of the interrogator. In these embodiments, the interrogator can compute an adjustment to a position of the focal point that counters the measured movement. For example, by computing and applying this adjustment, the interrogator can compensate for small movements of the interrogator operator's hand by electronically steering the ultrasound beam such that a net change of the absolute position of the focal point remains close to zero.

In step 1114, the interrogator enters a signal tracking state to increase alignment of the emitted US beam with the implantable device. In some embodiments, step 1114 corresponds to step 910 of FIG. 9 and step 1010 of FIG. 10. In some embodiments, step 1114 include steps 1116-1118.

In step 1116, the interrogator estimates a location of the implantable device based on the received ultrasonic backscatter.

In step 1118, the interrogator emits the US beam to focus on a focal point closer to the estimated location. As described above, the interrogator may use receive beam forming to determine a direction to adjust the focal point and increment the focal point in the determined direction. As described above with respect to FIGS. 9-10, once the implantable device has been tracked, the interrogator can re-establish the signal-steady state with the implantable device.

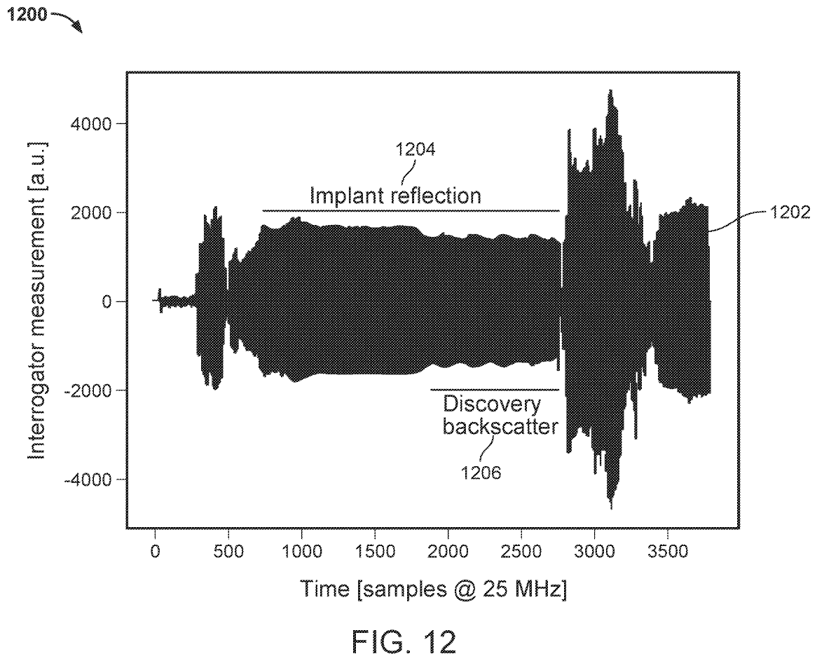

FIG. 12 illustrates an example diagram 1200 showing a predetermined pattern encoded by an implantable device into an ultrasonic backscatter 1202 received by an interrogator, according to some embodiments. As shown in diagram 1200, ultrasonic backscatter 1202 has varying amplitude across time (sampled at 25 MHz).

As described above, when an interrogator emits an US beam at the implantable device, ultrasonic waves within the US beam are reflected in the form of ultrasonic backscatter. Ultrasonic backscatter 1202 can include a portion 1204 depicting an implant reflection of ultrasonic waves and a portion 1206 depicting a waveform pattern embedded by the implantable device within ultrasonic backscatter 1202. In some embodiments, as described above with respect to FIG. 7, the interrogator can compare ultrasonic backscatter 1202 with a predetermined pattern associated with the implantable device to determine if it matches the predetermined pattern expected of the implantable device being powered on.

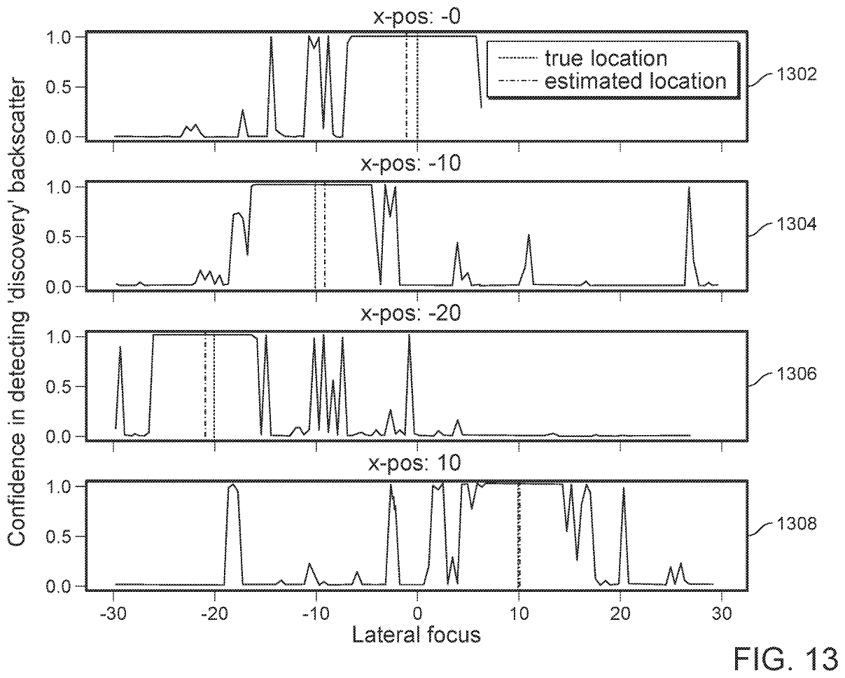

FIG. 13 illustrates example charts 1302-1308 that show how accurately an interrogator estimates a location of an implantable device in a discovery mode, according to some embodiments. In four experimental setups, the interrogator was configured to perform method 700 of FIG. 7. As shown in charts 1302-1308, a confidence level determined by the interrogator for whether the predetermined pattern of the implantable device is detected in ultrasonic backscatter across a plurality of focal points, shown as lateral focus, ranges from 0.0 to 1.0.

In some embodiments, the interrogator can be configured to apply statistical measures to the focal points at which the predetermined pattern of the implantable device is detected with a confidence above a threshold (e.g., 80%, 90%, 95%, etc.) to determine an estimate location of the implantable device. In the example charts 1302-1308, the interrogator was configured to calculate a spectral centroid (i.e., the center of mass) of the confidence levels (also referred to as a “score”) across the lateral focus range of focal points. As shown in FIG. 13, the interrogator's estimated locations in each of the four experimental setups was close to the true location of the implantable devices.

FIG. 14 illustrates a diagram 1400 of an implantable device 1411 configured to interact with a nerve 1414 of a subject, according to some embodiments. In some embodiments, implantable device 1411 can be an example implementation of implantable device 120 or 604 as described above with respect to FIGS. 1 and 6, respectively. As shown in diagram 1400, implantable device 1411 can be implanted on nerve 1414 and include one or more curved member such as curved member 1402 extending from a body 1412. Body 1412 of implantable device 1411 can include integrated circuit 1424 (including, e.g., modulation and demodulation circuit 612, stimulation circuit 614, detection circuit 616, or controller circuit 620), a non-transitory memory 1426 (e.g., memory 680), a power circuit 1428 (e.g., power circuit 630), and an ultrasonic transducer 1430 (e.g., ultrasonic transducer 608 or ultrasonic transducer circuit 606). In some embodiments, body 1412 includes a plurality of ultrasonic transducers including ultrasonic transducer 1430. Accordingly, it is to be understood that ultrasonic transducer 1430, as shown in diagram 800, may represent a plurality of ultrasonic transducers.

In some embodiments, ultrasonic transducer 1430 can be configured to receive ultrasonic waves transmitted by an interrogator (e.g., interrogator 106 of FIG. 1 or interrogator 502 of FIG. 5) and convert the mechanical energy of the ultrasonic waves into an electrical signal having an electrical energy. In some embodiments, the ultrasonic waves can include one or more operating mode commands that are detected by integrated circuit 1424 to set an operating mode of implantable device 1411 to one operating mode from a plurality of operating modes. In some embodiments, the electrical signal includes electrical representations of the one or more operating mode commands.

In some embodiments, a portion of the electrical signal can be processed by power circuit 1428 to power the components of implantable device 1411. In some embodiments, power circuit 1428 can include a power conveyor circuit (e.g., power conveyor circuit 634) configured to convert the electrical signal having a first voltage to a second signal having a second voltage to power various components of integrated circuit 1424. In some embodiments, power circuit 1428 can include a rectifying circuit (e.g., an active rectifier) to convert the electrical signal in an AC form to a DC form where the converted electrical signal may be associated with the first voltage. In some embodiments, the power conveyor circuit can include a charge pump to generate the second voltage greater than the first voltage. In some embodiments, power circuit 1428 can include an energy storage device (e.g., energy storage device 636) configured to store excess energy provided by the electrical signal and to operate as a secondary power source if the power supplied by the interrogator is insufficient. In some embodiments, the power conveyor circuit can be configured to control whether power is to be conveyed to or from the energy storage device, which effectively charges or discharges the energy storage device, respectively. In some embodiments, the power conveyor circuit can be configured control an amount of time (e.g., a number of clock cycles) that the power is conveyed in addition to the direction of power flow (e.g., in forward flow or in reverse flow).

In some embodiments, integrated circuit 1424 includes a controller circuit (e.g., controller circuit 620) configured to set the operating mode of implantable device 1411 based on an operating mode command received in the ultrasonic waves.

In some embodiments, the operating mode command can instruct implantable device 1411 to enter a power synchronization mode in which the controller circuit can generate information indicating implantable device 1411. For example, integrated circuit 1424 may be configured to modulate an electric signal to embed a predetermined pattern within an ultrasonic backscatter emitted by implantable device 1411. As described above with respect to FIGS. 1-13, an interrogator receiving the ultrasonic backscatter can extract the predetermined pattern to discover or track a location of implantable device 1411. By adjusting a beam focus of an emitted US beam, the interrogator can more effectively align the US beam with implantable device 1411 to maintain sufficient power supplied to implantable device 1411. Moreover, since the US beam emitted by the interrogator is used both to power and to communicate with implantable device 1411, maintaining sufficient power also improves ultrasonic communications between the interrogator and implantable device 1411.

In some embodiments, the operating mode command can instruct implantable device 1411 to enter a nerve-stimulation mode or a detection mode, each of which may operate electrode pads 1418 on curved member 1402. In some embodiments, the detection mode may be an example of an uplink mode associated with transmitting device data to other devices such as the interrogator. In some embodiments, in the detection mode, electrode pads 1418 are configured to detect an electrophysiological signal, and a detection signal based on the electrophysiological signal is received by integrated circuit 1424. The detection signal received by integrated circuit 1424 may be processed (for example, amplified, digitized, and/or filtered) by a detection circuit (e.g., by detection circuit 616) before being received by the controller circuit. In some embodiments, the controller circuit can access non-transitory memory (e.g., memory 680) to store data related to the detected electrophysiological signal. In some embodiments, in the detection mode, the controller circuit can be configured to operate ultrasonic transducer 1430 to emit a backscatter of received ultrasonic waves in which the backscattered ultrasonic waves encodes the data related to the detected electrophysiological signal.

In some embodiments, the operating mode command can instruct implantable device 1411 to enter the nerve-stimulating mode. In the stimulation mode, the controller circuit can generate a stimulation signal based on the detection signal, and operate one or more electrode pads 1418 to emit an electrical pulse to nerve 1414 based on the stimulation signal. In some embodiments, the controller circuit can access the non-transitory memory (e.g., memory 680) to store data related to the stimulation signal or electrical pulse emitted to nerve 1414. In some embodiments, in the stimulation mode, the controller circuit can be configured to operate ultrasonic transducer 1430 to emit a backscatter of received ultrasonic waves in which the backscattered ultrasonic waves encodes data related a status of the stimulation.

Data stored on the non-transitory memory can be wirelessly transmitted through ultrasonic backscatter waves emitted by ultrasonic transducer 1430. As described above with respect to FIG. 6, to transmit data using the ultrasonic backscatter, ultrasonic transducer 1430 may first receive ultrasonic waves and generate an electrical current that flows through a modulation circuit. Then, the controller circuit may access the memory and operate the modulation circuit to modulate the electrical current flowing through the modulation circuit to encode the data. Through such a process, the ultrasonic backscatter waves emitted by ultrasonic transducer 1430 can encode the data.

In some embodiments, as shown in diagram 1400, curved member 1402 can include a first portion 1402a and a second portion 1402b bridged by body 1412 at point 1416. In some embodiments, first portion 1402a and second portion 1402b are directly connected, and curved member 1402 is attached to body 1412 through a connecting member. Curved member 1402 can include a plurality of electrode pads 1418 on the inner surface of curved member 1402, and electrode pads 1418 can be radially positioned around an axis parallel to the length of nerve 1414. A separation 1420 between first portion 1402a and second portion 1402b is present along curved member 1402 (which may be similarly present in other curved members of implantable device 1411). In some embodiments, implantable device 411 can be implanted by flexing first portion 1402a and second portion 1402b of curved member 1402 outwardly, thereby expanding the size of the separation and allowing nerve 1414 or other filamentous tissue to pass through separation 1420 and fit within the cylindrical space formed by curved member 1402. First portion 1402a and second portion 1402b of curved member 1402 can be released, which allows curved member 1402 to wrap around nerve 1414 or other filamentous tissue.

The plurality of electrode pads 1418 of as shown in FIG. 14 are outside of nerve 1414, but in direct contact with the epineurium of nerve 1414. Nerve 1414 can include several fascicles 1422. In some embodiments, electrode pads 1418 within curved member 1402 can be operated for targeted emission of an electrical pulse to one or more of fascicles 1422 or other subset of nerve fibers, and/or operated for targeted detection of an electrophysiological signal transmitted by one or more of fascicles 1422 or other subset of nerve fibers. For example, electrode pads 1418 can be selectively activated by the controller circuit within integrated circuit 1424, which is housed within body 1412, to emit an electric pulse targeted to one or more fascicles 1422. In another example, electrode pads 418 are operated by the controller circuit to detect an electrophysiological signal transmitted by one or more of fascicles 1422 within nerve 1414. In some embodiment, curved member 1402 can be configured to detect the electrophysiological signal transmitted by nerve 1414 or a subset of nerve fibers, emit an electrical pulse to nerve 1414 or targeted to a subset of nerve fibers, or both detect the electrophysiological signal transmitted by nerve 1414 or a subset of nerve fibers and emit an electrical pulse to nerve 1414 or targeted to a subset of nerve fibers. For example, implantable device 1411 may include a plurality of curved members (including curved member 1402) in which a first curved member can be configured to detect the electrophysiological signal transmitted by nerve 1414 or a subset of nerve fibers, and a second curved member can be configured to emit an electrical pulse to nerve 1414 or targeted to a subset of nerve fibers.

In some embodiments, curved member 1402 can be sized to engage a selected nerve 1414 or fibrous tissue containing nerve 1414. Nerve 1414 can be the spinal cord or a peripheral nerve. In some embodiments, nerve 414 is an autonomic nerve or a somatic nerve. In some embodiments, nerve 414 is a sympathetic nerve or a parasympathetic nerve. In some embodiments, nerve 1414 is a vagus nerve, a mesenteric nerve, a splenic nerve, a sciatic nerve, a tibial nerve, a pudendal nerve, a celiac ganglion, a sacral nerve, or any branch thereof.

The size, shape, and spacing of curved member 1402 on implantable device 1411 can depend on the type and size of tissue that implantable device 1411 engages. In some embodiments, two or more curved members of implantable device 1411 are spaced by about 0.25 mm or more (such as about 0.5 mm or more, about 1 mm or more, about 2 mm or more, about 3 mm or more, about 4 mm or more, about 5 mm or more, about 6 mm or more, or about 7 mm or more). In some embodiments, the two or more curved members are space by about 8 mm or less (such as about 7 mm or less, about 6 mm or less, about 5 mm or less, about 4 mm or less, about 3 mm or less, about 2 mm or less, about 1 mm or less, or about 0.5 mm or less). By way of example, the two or more curved members can be spaced about 0.25 mm to about 0.5 mm, about 0.5 mm to about 1 mm, about 1 mm to about 2 mm, about 2 mm to about 3 mm, about 3 mm to about 4 mm, about 4 mm to about 5 mm, about 5 mm to about 6 mm, about 5 mm to about 7 mm, or about 7 mm to about 8 mm apart. The width of curved member 1402 can also vary depending on the application of implantable device 1411 or the tissue engaged by implantable device 1411. In some embodiments, the width of curved member 1402 is about 100 μm or more (such as about 150 μm or more, about 250 μm or more, about 500 μm or more, about 1 mm or more, or about 1.5 mm or more). In some embodiments, the width of curved member 1402 is about 2 mm or less (such as about 1.5 mm or less, about 1 mm or less, about 500 μm or less, about 250 μm or less, or about 150 μm or less. In some embodiments, the width of curved members 1402 is about 100 μm to about 2 mm (such as about 100 μm to about 150 μm, about 150 μm to about 250 μm, about 250 μm to about 500 μm, about 500 μm to about 1 mm, about 1 mm to about 1.5 mm, or about 1.5 mm to about 2 mm). The inner surface of curved member 1402 form a cylindrical space through which nerve 414 and/or filamentous tissue passes. The diameter of the cylindrical space formed by curved member 402 depends on the target nerve and/or filamentous tissue that implantable device 1411 will engage. In some embodiments, curved member 1402 forms a cylindrical space with a diameter of about 50 μm to about 15 mm (for example, about 50 μm to about 100 μm, about 100 μm to about 250 μm, about 250 μm to about 500 μm, about 500 μm to about 1 mm, about 1 mm to about 1.5 mm, about 1.5 mm to about 2.5 mm, about 2.5 mm to about 5 mm, about 5 mm to about 10 mm, or about 10 mm to about 15 mm).

In some embodiments, implantable device 1411 includes one or more additional securing members configured to secure implantable device 1411 to the filamentous tissue. Such securing members can include, for example, loops for suturing the implantable device to anatomical structure (such as the filamentous tissue or nerve, or other tissue surrounding the filamentous tissue or nerve), pins, or clamps. For example, implantable device 1411 can be sutured to the filamentous tissue or nerve 1414, or tissue surrounding the filamentous tissue or nerve, to limit movement of implantable device 411 once implanted.

In some embodiment, curved member 1402 of implantable device 1411 can include a metal, metal alloy, ceramic, silicon, or a non-polymeric material. Curved member 1402 may be flexible, and is preferably sprung such that curved member 1402 can be positioned around nerve 1414 and/or filamentous tissue. In some embodiments, curved member 1402 or a portion of curved member 402 is coated with an elastomeric coating or a non-elastomeric coating, which is preferably bioinert, such as polydimethylsioloxane (PDMS), a silicone, a urethane polymer, a poly(p-xylylene)polymer (such as a poly(p-xylylene) polymer sold under the tradename PARYLENE®), or a polyimide. Curved member 1402 can include a plurality of electrode pads 1418 on an inner surface. In some embodiments, electrode pads 1418 on the inner surface of curved member 1402 are not coated with the elastomeric coating or the non-elastomeric polymer coating, although the inner surface may be coated with a conductive material (e.g., electroplated with a PEDOT polymer or a metal to improve electrical characteristics of the electrode pad). Accordingly, in some embodiments, only the outer surface of curved member 402 is coated with the coating. Optionally, the coating further coats the housing of body 1412.

In some embodiments, the plurality of electrode pads 1418 can include 3, 4, 5, 6, 7, 8, 9, 10, 11, 12, 13, 14, 15, 16, 17, 18, 19, 20, or more electrode pads, such as between about 3 and about 50 electrode pads, between about 3 and about 5 electrode pads, between about 5 and about 10 electrode pads, between about 10 and about 25 electrode pads, or between about 25 and about 50 electrode pads. In some embodiments, the electrode pads within the plurality of electrode pads 1418 can be selectively activated by the controller circuit, which allows for targeted electrical pulse emission, as further described herein.

In some embodiments, electrode pads 1418 can include any suitable conductive material, such as one or more of (or an alloy of one or more of) tungsten, platinum, palladium, gold, iridium, niobium, tantalum, or titanium. The material of the detecting electrode pads and the stimulating electrode pads may be the same or different. The size and shape of electrode pads 1418 may also be the same or different. For example, electrode pads 1418 on a given curved member 1402 may be of the same or different size, and electrode pads on different curved members may be of the same or different size.

In some embodiments, electrode pads 1418 of implantable device 1411 are positioned by curved member 1402 to be in electrical communication with nerve 1414. In some embodiments, electrode pads 1418 are not in direct contact with nerve 1414 (for example outside and not indirect contact with nerve 1414), but are in electrical communication with nerve 1414. In some embodiments, electrode pads 1418 are positioned within about 2 mm (e.g., within about 1.8 mm, within about 1.6 mm, within about 1.4 mm, within about 1.2 mm, within about 1.0 mm, within about 0.8 mm, within about 0.6 mm, within about 0.4 mm, or within about 0.2 mm) of nerve 1414. In some embodiments, electrode pads 1418 are configured to penetrate the epineurium of nerve 1414 at one or more locations. For example, electrode pads 1418 can be needle-shaped, which allows for penetration of the epineurium. In some embodiments, electrode pads 818 directly contact nerve 1414, for example the epineurium of nerve 1414.

In some embodiments, body 1412 includes a housing, which can include a base, one or more sidewalls, and a top. The housing can enclose ultrasonic transducer 1430 and integrated circuit 1424. The housing may be sealed closed (for example by soldering or laser welding) to prevent interstitial fluid from coming in contact with ultrasonic transducer 1430 or integrated circuit 1424. The housing is preferably made from a bioinert material, such as a bioinert metal (e.g., steel or titanium) or a bioinert ceramic (e.g., titania or alumina). The housing (or the top of the housing) may be thin to allow ultrasonic waves to penetrate through the housing. In some embodiments, the thickness of the housing is about 100 micrometers (μm) or less in thickness, such as about 75 μm or less, about 50 μm or less, about 25 μm or less, or about 10 μm or less. In some embodiments, the thickness of the housing is about 5 μm to about 10 μm, about 10 μm to about 25 μm, about 25 μm to about 50 μm, about 50 μm to about 75 μm, or about 75 μm to about 100 μm in thickness.

In some embodiments, body 1412 of implantable device 1411 is relatively small, which allows for comfortable and long-term implantation while limiting tissue inflammation that is often associated with implantable medical devices. In some embodiments, the longest dimension of body 1412 is about 10 mm or less, such as about 5 mm to about 9 mm, or about 6 mm to about 8 mm. For example, the longest dimension may be a length or a height of body 1412 of implantable device 1411. In some embodiments, the longest width of body 1412 is about 5 mm or less, such as about 2 mm to 5 mm, or about 3 mm to 4 mm.

In some embodiments, body 1412 includes a material, such as a polymer, within the housing. The material can fill empty space within the housing to reduce acoustic impedance mismatch between the tissue outside of the housing and within the housing. Accordingly, body 1412 is preferably void of air or vacuum, according to some embodiments.

In some embodiments, ultrasonic transducer 1430 can include a micro machined ultrasonic transducer, such as a capacitive micro-machined ultrasonic transducer (CMUT) or a piezoelectric micro-machined ultrasonic transducer (PMUT), or can include a bulk piezoelectric transducer. Bulk piezoelectric transducers can be any natural or synthetic material, such as a crystal, ceramic, or polymer. Example bulk piezoelectric transducer materials may include barium titanate (BaTiO₃), lead zirconate titanate (PZT), zinc oxide (ZO), aluminum nitride (AlN), quartz, berlinite (AlPO₄), topaz, langasite (La₃Ga₅SiO₁₄), gallium orthophosphate (GaPO₄), lithium niobate (LiNbO₃), lithium tantalite (LiTaO₃), potassium niobate (KNbO₃), sodium tungstate (Na₂WO₃), bismuth ferrite (BiFeO₃), polyvinylidene (di)fluoride (PVDF), and lead magnesium niobate-lead titanate (PMN-PT).

In some embodiments, the bulk piezoelectric transducer is approximately cubic (i.e., an aspect ratio of about 1:1:1 (length:width:height)). In some embodiments, the piezoelectric transducer is plate-like, with an aspect ratio of about 5:5:1 or greater in either the length or width aspect, such as about 7:5:1 or greater, or about 10:10:1 or greater. In some embodiments, the bulk piezoelectric transducer is long and narrow, with an aspect ratio of about 3:1:1 or greater, with the longest dimension being aligned to the direction of the ultrasonic backscatter waves (i.e., the polarization axis). In some embodiments, one dimension of the bulk piezoelectric transducer is equal to one half of the wavelength (k) corresponding to the drive frequency or resonant frequency of the transducer. At the resonant frequency, the ultrasound wave impinging on either the face of the transducer will undergo an 180° phase shift to reach the opposite phase, causing the largest displacement between the two faces. In some embodiments, the height of the piezoelectric transducer is about 10 μm to about 1000 μm (such as about 40 μm to about 400 μm, about 100 μm to about 250 μm, about 250 μm to about 500 μm, or about 500 μm to about 1000 μm). In some embodiments, the height of the piezoelectric transducer is about 5 mm or less (such as about 4 mm or less, about 3 mm or less, about 2 mm or less, about 1 mm or less, about 500 μm or less, about 400 μm or less, 250 μm or less, about 100 μm or less, or about 40 μm or less). In some embodiments, the height of the piezoelectric transducer is about 20 μm or more (such as about 40 μm or more, about 100 μm or more, about 250 μm or more, about 400 μm or more, about 500 μm or more, about 1 mm or more, about 2 mm or more, about 3 mm or more, or about 4 mm or more) in length.

In some embodiments, ultrasonic transducer 1430 has a length of about 5 mm or less (such as about 4 mm or less, about 3 mm or less, about 2 mm or less, about 1 mm or less, about 500 μm or less, about 400 μm or less, 250 μm or less, about 100 μm or less, or about 40 μm or less) in the longest dimension. In some embodiments, ultrasonic transducer 1430 has a length of about 20 μm or more (such as about 40 μm or more, about 100 μm or more, about 250 μm or more, about 400 μm or more, about 500 μm or more, about 1 mm or more, about 2 mm or more, about 3 mm or more, or about 4 mm or more) in the longest dimension.

In some embodiments, ultrasonic transducer 1430 is connected to two electrodes to allow electrical communication with integrated circuit 1424. The first electrode is attached to a first face of ultrasonic transducer 1430 and the second electrode is attached to a second face of ultrasonic transducer 1430, with the first face and the second face on opposite sides of ultrasonic transducer 1430 along one dimension. In some embodiments, the electrodes include silver, gold, platinum, platinum-black, poly(3,4-ethylenedioxythiophene (PEDOT)), a conductive polymer (such as conductive PDMS or polyimide), or nickel. In some embodiments, the axis between the electrodes of ultrasonic transducer 1430 is orthogonal to the motion of ultrasonic transducer 1430.

The foregoing description sets forth exemplary methods, parameters and the like. It should be recognized, however, that such description is not intended as a limitation on the scope of the present disclosure but is instead provided as a description of exemplary embodiments. The illustrative embodiments described above are not intended to be exhaustive or to limit the disclosure to the precise forms disclosed. Many modifications and variations are possible in view of the above teachings. The embodiments were chosen and described to best explain the principles of the disclosed techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. In the foregoing description of the disclosure and embodiments, reference is made to the accompanying drawings, in which are shown, by way of illustration, specific embodiments that can be practiced. It is to be understood that other embodiments and examples can be practiced, and changes can be made without departing from the scope of the present disclosure.

Although the foregoing description uses terms first, second, etc. to describe various elements, these elements should not be limited by the terms. These terms are only used to distinguish one element from another.

Reference to “about” or “approximately” a value or parameter herein includes (and describes) variations that are directed to that value or parameter per se. For example, description referring to “about X” includes description of “X.”

It is understood that aspects and variations of the invention described herein include “consisting” and/or “consisting essentially of” aspects and variations.

The terms “implantable” and “implanted” refer to an object being fully implantable or fully implanted in a subject such that no portion of the object breaches the surface of the subject.

The term “substantially” refers to 90% or more. For example, a curved member that substantially surrounds a cross-section of a nerve refers to a curved member that surrounds 90% or more of the cross-section of the nerve.

The term “subject” and “patient” are used interchangeably herein to refer to a vertebrate animal such as a human.

The terms “treat,” “treating,” and “treatment” are used synonymously herein to refer to any action providing a benefit to a subject afflicted with a disease state or condition, including improvement in the condition through lessening, inhibition, suppression, or elimination of at least one symptom, delay in progression of the disease or condition, delay in recurrence of the disease or condition, or inhibition of the disease or condition.

Where a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

In addition, it is also to be understood that the singular forms “a,” “an,” and “the” used in the foregoing description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

The term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

Features and preferences described above in relation to “embodiments” are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

What is claimed is:

1. A method for tracking an implantable device powered using ultrasonic waves to maintain power supplied to the implantable device, comprising:
establishing a synchronization state with the implantable device, comprising:
emitting an ultrasonic beam to a first focal point and receiving a first ultrasonic backscatter corresponding to the emitted ultrasonic beam;
determining a first signal strength based on the first ultrasonic backscatter; and
establishing the synchronization state with the implantable device in response to determining that the first signal strength is at or above a predetermined threshold;
estimating a location of the implantable device;
emitting the ultrasonic beam to a second focal point closer to the estimated location than the first focal point and receiving second ultrasonic backscatter corresponding to the emitted ultrasonic beam;
determining a second signal strength based on the second ultrasonic backscatter; and
determining whether to maintain or to adjust where the emitted ultrasonic beam is being focused based on comparing the determined second signal strength with the first signal strength.

2. The method of claim 1, wherein establishing the synchronization state comprises controlling the ultrasonic beam to successively focus on a plurality of focal points in a search region to determine the first focal point at which the first signal strength meets the predetermined threshold.

3. The method of claim 2, wherein controlling the ultrasonic beam comprises directing the ultrasonic beam in a first direction to successively focus on the plurality of focal points until the first signal strength determined from the first ultrasonic backscatter is determined to be above the predetermined threshold.

4. The method of any one of claims 1-3, comprising, in response to determining to maintain the determined focal point of the ultrasonic beam at the second focal point:
maintaining the ultrasonic beam to focus on the determined second focal point, and
monitoring a signal strength determined from ultrasonic backscatter received while the ultrasonic beam is focused on the determined second focal point.

5. The method of claim 4, wherein the monitored signal strength corresponds to a modulated signal generated by the implantable device to encode information into ultrasonic backscatter received at an interrogator.

6. The method of claim 5, wherein the encoded information uniquely identifies the implantable device.

7. The method of any one of claims 1-3, comprising, in response to determining to adjust the second focal point of the ultrasonic beam, iteratively estimating the location of the implantable device based on received ultrasonic backscatter and updating a focal point of the ultrasonic beam in the direction of the estimated location until a signal strength determined from ultrasonic backscatter received for the updated focal point is no longer increasing.

8. The method of any one of claims 1-7, wherein determining the first signal strength based on the first ultrasonic backscatter comprises:
extracting, from the first ultrasonic backscatter, an implant signal associated with the implantable device; and
determining the first signal strength based on the extracted implant signal.

9. The method of claim 8, wherein extracting the implant signal comprises cancelling signal interferences from the backscattered ultrasonic waves to extract the implant signal.

10. The method of claim 9, comprising identifying the implantable device being tracked based on the extracted implant signal.

11. The method of any one of claims 1-10, wherein the first ultrasonic backscatter comprises a first portion that includes an implant signal encoded by the implantable device into the first ultrasonic backscatter, and a second portion that does not include the implant signal.

12. The method of claim 11, comprising determining the first signal strength of the implant signal based on comparing the first portion and the second portion of the first ultrasonic backscatter.

13. The method of any one of claims 1-12, wherein the location of the implantable device is estimated after establishing the synchronization state.

14. The method of any one of claims 1-13, wherein the location of the implantable device is estimated based on receive beamforming.

15. The method of any one of claims 1-14, comprising determining a focal point associated with a local maximum signal strength comprising, iteratively:
estimating the location of the implantable device;
directing the ultrasonic beam from a current focal point to a test focal point based on a direction of the estimated location of the implantable device relative to the current focal point, wherein the current focal point becomes a previous focal point;
determining a signal strength based on ultrasonic backscatter when the ultrasonic beam is emitted to the test focal point; and
comparing the signal strength when the ultrasonic beam is emitted to the test focal point to the signal strength when the ultrasonic beam is emitted to the previous focal point.

16. The method of claim 15, comprising, in response to determining the focal point associated with the local maximum, establishing a steady state with the implantable device, wherein, if the signal strength decreases below a second predetermined threshold, the focal point associated with the local maximum signal is re-determined.

17. The method of any one of claims 1-16, wherein determining whether to maintain where the emitted ultrasonic beam is being focused comprises:
monitoring a movement of an interrogator; and
determining an adjustment to a focal point of the ultrasonic beam based on the monitored movement.

18. The method of any one of claims 1-17, wherein the method for tracking the implantable device is performed at an interrogator device.

19. A system for tracking an implantable device powered using ultrasonic waves, comprising:
a transducer array comprising a plurality of transducers; and
a controller configured to:
establish a synchronization state with the implantable device, comprising:
control the transducer array to emit an ultrasonic beam to a first focal point and receive a first ultrasonic backscatter corresponding to the emitted ultrasonic beam;
determine a first signal strength based on the first ultrasonic backscatter; and
establish the synchronization state with the implantable device in response to determining that the first signal strength is at or above a predetermined threshold;
estimate a location of the implantable device;
control the transducer array to emit the ultrasonic beam to a second focal point closer to the estimated location than the first focal point and receiving second ultrasonic backscatter corresponding to the emitted ultrasonic beam;
determine a second signal strength based on the second ultrasonic backscatter; and
determine whether to maintain or to adjust where the emitted ultrasonic beam is being focused based on comparing the determined second signal strength with the first signal strength.

20. A method for discovering an implantable device powered using ultrasonic waves, comprising:
emitting an ultrasonic beam to successively focus on a plurality of focal points;
at each focal point of the plurality of focal points:
holding the focused ultrasonic beam at the focal point for a duration that permits the implantable device, if located at the focal point, to convert energy from ultrasonic waves of the ultrasonic beam into electrical energy to enter into a powered-on state from a powered-off state,
receiving an ultrasonic backscatter corresponding to the ultrasonic beam focused on the focal point, and
comparing the received ultrasonic backscatter with a predetermined pattern associated with the implantable device to be discovered to generate a score indicating how likely the ultrasonic backscatter comprises the predetermined pattern; and
determining a location of the implantable device from the plurality of focal points based on a plurality of scores generated for each focal point within the plurality of focal points.

21. The method of claim 20, comprising causing the implantable device to enter into the powered-on state.

22. The method of claim 20 or 21, further comprising establishing an ultrasonic communication link with the implantable device using ultrasonic waves emitted by the interrogator focused at the focal point corresponding to the determined location of the implantable device.

23. The method of any one of claims 20-22, wherein the plurality of focal points corresponds to a steerable range of the ultrasonic beam.

24. The method of any one of claims 20-23, wherein the predetermined pattern comprises one or more square waves.

25. The method of any one of claims 20-24, wherein the predetermined pattern uniquely identifies the implantable device.

26. The method of any one of claims 20-25, wherein the predetermined pattern comprises information encoded by the implantable device into the ultrasonic backscatter.

27. The method of claim 26, wherein the implantable device receives the ultrasonic waves from the emitted ultrasonic beam and encodes the information into the ultrasonic backscatter by modulating an electric signal generated based on the ultrasonic waves received at the implantable device.

28. The method of any one of claims 20-27, wherein determining the location of the implantable device comprises selecting a focal point from a subset of focal points within the plurality of focal points, wherein the score corresponding to each focal point within the subset of focal points is above a predetermined threshold value.

29. The method of any one of claims 20-27, wherein determining the location of the implantable device comprises selecting a focal point from the plurality of focal points as being the most likely location of the implantable device based the plurality of scores.

30. The method of claim 28 or 29, comprising confirming the location of the implantable device, comprising:
emitting the ultrasonic beam to focus on the selected focal point for a predetermined time period; and
analyzing an ultrasonic backscatter received while the ultrasonic beam is focused on the selected focal point to confirm that the implantable device is located at the selected focal point.

31. The method of claim 30, comprising, in response to confirming that the implantable device is located at the selected focal point, maintaining the ultrasonic beam at the selected focal point.

32. The method of any one of claims 20-31, wherein the method for discovering the implantable device is performed at an interrogator device.

33. The method of claim 32, wherein the interrogator comprises a plurality of transducers in a transducer array, and wherein emitting the ultrasonic beam to successively focus on the plurality of focal points comprises controlling the plurality of transducers to transmit ultrasonic waves in the ultrasonic beam to successively focus on the plurality of focal points.

34. The method of claim 33, wherein emitting the ultrasonic beam comprises successively directing the focused ultrasonic beam at each focal points of the plurality of focal points in a steerable angular range of the transducer array.

35. The method of claim 33, wherein emitting the ultrasonic beam comprises mechanically moving the transducer array to successively direct the focused ultrasonic beam at each focal points of the plurality of focal points.

36. The method of any one of claims 33-35, wherein emitting the ultrasonic beam comprises controlling when power is supplied to each transducer in the transducer array to successively direct the focused ultrasonic beam at each focal points of the plurality of focal points.

37. The method of any one of claims 20-36, wherein the implantable device comprises one or more capacitors to store the electrical energy converted from the ultrasonic waves of the ultrasonic beam to enter into the powered-on state from the powered-off state.

38. The method of any one of claims 1-18 and 20-37, wherein the ultrasonic beam has a spot size of less than 10 mm.

39. A system for discovering an implantable device powered using ultrasonic waves, comprising:
a transducer array comprising a plurality of transducers; and
a controller configured to:
control the transducer array to emit an ultrasonic beam successively focused on a plurality of focal point;
at each focal point of the plurality of focal points;
hold the focused ultrasonic beam at the focal point for a duration that permits the implantable device, if located at the focal point, to convert energy from ultrasonic waves of the ultrasonic beam into electrical energy and enter into a powered-on state from a powered-off state,
receive an ultrasonic backscatter corresponding to the emitted ultrasonic beam, and
compare the received ultrasonic backscatter with a predetermined pattern associated with the implantable device to be discovered to generate a score indicating how likely the ultrasonic backscatter comprise the predetermined pattern; and
determine a location of the implantable device from the plurality of focal points based on a plurality of scores generated for the plurality of corresponding focal points.
